(19) United States
(12) Patent Application Publication
(10) Pub. No.: US 2024/0000414 A1
(43) Pub. Date: Jan. 4, 2024

(54) METHOD AND SYSTEM FOR SEGMENTING AND CHARACTERIZING AORTIC TISSUES

(71) Applicant: VITAA MEDICAL SOLUTIONS INC., Montreal (CA)

(72) Inventors: Atefeh Abdolmanafi, Newmarket (CA); Elena Di Martino, Calgary (CA); Arianna Forneris, Edmonton (CA)

(21) Appl. No.: 18/035,151

(22) PCT Filed: Feb. 22, 2022
(86) PCT No.: PCT/IB2022/051558

§ 371 Date: May 3, 2023

Related U.S. Application Data
(60) Provisional application No. 63/152,105, filed on Feb. 22, 2021.

(51) Int. Cl. A61B 6/00; G06T 5/00; G06T 7/00; G06V 10/25; G06V 10/44; G06V 10/82; G06V 10/26; G06V 20/50; G06V 10/764

(52) U.S. Cl. CPC A61B 6/5217; A61B 6/504; G06T 5/001; G06T 7/0012; G06V 10/25; G06V 10/44; G06V 10/82; G06V 10/26; G06V 20/50; G06V 10/764; A61B 6/03

(57) ABSTRACT

A method for segmenting aortic tissues (or detecting calcification) in an image of a body of a given subject, the method executed by a processor having access to at least one deep learning model trained to segment aortic tissues in images, the method including: receiving the image of the body of the given subject, the image being an aorta, an intraluminal thrombus and additional body parts; extracting a region of interest from the received image, the region of interest being the aorta and the intraluminal thrombus; determining, within the region of interest, a presence of a calcification on at least one of an aortic wall and the intraluminal thrombus; and outputting an indication of the presence of the calcification.

### FIELD

The present technology pertains to the field of medical imaging. More specifically, the present technology relates to a method and a system for segmenting and characterizing aortic tissues in images by using trained machine learning models.

### BACKGROUND

Aortic aneurysm (AA) is a focal dilation of the aorta, which results in progression and rupture if it is not diagnosed and subsequently treated. It is a major cause of mortality and morbidity worldwide. AA is caused by a failure of the major structural proteins (elastin and collagen) in the aorta. It mostly occurs after media degeneration (i.e., degeneration of the arterial second layer) and leads to widening of the lumen and loss of structural integrity. When the maximum aortic diameter exceeds the normal diameter, it is considered an aneurysm, which is assessed using Computed Tomography (CT) imaging. Lack of accurate diagnosis and treatment results in progressive dilatation and rupture when the aneurysm cannot tolerate the luminal blood pressure. Therefore, risk assessment of ruptures and adverse events has an important role in the determination of the clinical course for patients suffering from AA. Currently, the assessment is done by measuring the aneurysm diameter using CT, MR or Ultrasound diagnostic images of the aorta. According to the Society of Vascular Surgery practice guidelines, there is a lack of standardization in determining disease progression and there is a considerable variability in measuring the aortic diameter since it is performed either manually or semi-automatically with human intervention. Thus, the lack of automated aneurysm segmentation tools is a limitation to be addressed.

There is a need for methods and systems for segmenting aortic and aneurysm tissues to determine the degree and the rate of the disease progression. Furthermore, it is important to detect aortic tissues accurately to evaluate changes after each patient's follow up and linking those changes to further structural degeneration which would enable an accurate estimation of the biomechanical properties of various aortic tissues, as well as accurate identification and detection of the lumen when a contrast medium is not used during image acquisition.

Further, identification of calcifications in the aortic wall tissues may improve the rupture risk assessment of AA. Intraluminal thrombus (ILT) is present in the majority of abdominal aortic aneurysms. The size, presence of fissures, dissections, and calcifications in the ILT are important attributes, which can contribute to aneurysm progression and increased rupture risk.

### SUMMARY

It is an object of the present technology to ameliorate at least some of the inconveniences present in the prior art. One or more embodiments of the present technology may provide and/or broaden the scope of approaches to and/or methods of achieving the aims and objects of the present technology.

One or more embodiments of the present technology have been developed based on the inventors' appreciation that improved patient outcomes depend on the accurate diagnosis and evaluation of the aortic wall in aortic disease. Understanding the location, progression, and characteristics of the pathological formations caused by aortic disease(s) including aneurysms, intraluminal thrombus, and calcifications is only possible through imaging of the aortic wall in cross-sectional view. Various modalities are used to image the aortic wall. CT is a non-invasive diagnostic imaging system, which employs specialized x-rays to image the aorta. Current CT scanners have the spatial resolution of 0.625-2 mm in the z-axis and up to 0.5 mm in the x to y axes. Magnetic Resonance Imaging (MRI) is another modality that may be used for imaging AA. In contrast with CT, MRI does not use ionizing radiation, but is not as common due to its limited availability, higher cost, and lower spatial resolution (1-2 mm) than CT imaging.

More specifically, inventors of the present technology have appreciated that analyzing aortic CT images in AA present certain challenges, due to similarities in the pixel intensities of the aortic structures—including thrombus, aortic wall- and the periaortic tissues, which makes the manual extraction of the aorta challenging. If a contrast medium is not used during imaging, the lumen is generally not visually well recognizable. Current approaches to measure the aortic aneurysm diameter are either manual or semi-automatic and require human intervention. These factors render the task time-consuming, and error prone, due to differences in observations from one observer to another. Further, fluid dynamic analysis and evaluating the biomechanical properties of various arterial wall tissues require accurate detection and characterization of tissue types.

In some embodiment, the present technology contributes to the field by using fully convolutional networks (FCN) to leverage access to strong discriminating deep features which overcome limitations of patch-based convolutional neural networks (CNNs) used for segmentation tasks. Further, by using dilated convolutions instead of standard convolutions, the computational cost may be reduced, and model performance may be accelerated. Accurate recognition and extraction of the aorta can contribute to increasing the precision of algorithms to detect and quantify wall deformation by reducing errors and noisy results. Further, one or more embodiments of the present technology enable reproducibility of results minimizing human error in interpretation of the images.

Thus, one or more embodiments of the present technology are directed to a method of and a system for segmenting aortic tissues.

According to a first broad aspect, there is provided a method for segmenting aortic tissues in an image of a body of a given subject, the method being executed by a processor, the processor having access to at least one deep learning model having been trained to segment tissues in images, the method comprising: receiving the image of the body of the given subject, the image comprising an aorta, an intraluminal thrombus and additional body parts; extracting a region of interest from the received image, the region of interest comprising the aorta and the intraluminal thrombus; determining, within the region of interest, a presence of a calcification on at least one of an aortic wall and the intraluminal thrombus; and outputting an indication of the presence of the calcification.

In one embodiment, the at least one deep learning model comprises a first deep learning and a second deep learning model, said extracting the region of interest being performed by the first deep learning model and said determining the presence of the calcification is performed by the second deep learning model.

In one embodiment, the step of extracting the region of interest comprises: extracting, using the first deep learning model, first image features from the image, the first image features being indicative of the aorta and the intraluminal thrombus; and segmenting, using the first deep learning model, the region of interest from the image using the first image features.

In one embodiment, the first deep learning model comprises a fully convolutional network (FCN)-based model.

In one embodiment, the first deep learning model comprises dilated convolutional layers.

In one embodiment, the first deep learning model comprises a binary classifier.

In one embodiment, the method further comprises detecting an aortic lumen of the aorta within the region of interest, the aortic lumen, the aortic wall and the intraluminal thrombus forming together the region of interest.

In one embodiment, the image further comprises at least one artery, the at least one artery being part of the region of interest, and said detecting the aortic lumen further comprises detecting the artery lumen of the at least one artery.

In one embodiment, the at least one artery comprises at least one of: at least one common iliac artery, at least one internal iliac artery and at least one external iliac artery.

In one embodiment, the step of detecting the aortic lumen is performed by the second deep learning model, said detecting the aortic lumen and said determining the presence of the calcification being concurrently performed by the second deep learning model.

In one embodiment, the second deep learning model is configured for: extracting second image features from the region of interest, the second image features being indicative of the aortic lumen and the calcification, said determining the presence of the calcification being performed using the second image features.

In one embodiment, the second deep learning model comprises a ResNet-based fully conventional network model trained for multi-class segmentation.

In one embodiment, the at least one deep learning model further comprises a third deep learning model, said detecting the aortic lumen being performed by the third deep learning model, the method further comprising removing the aortic lumen from the region of interest, thereby obtaining the aortic wall and the intraluminal thrombus.

In one embodiment, the step of detecting the aortic lumen comprises: extracting, using the third deep learning model, second image features from the region of interest, the second image features being indicative of the aortic lumen; and segmenting, using the third deep learning model, the aortic lumen from the region of interest using the second image features; and wherein the second deep learning model is configured for: extracting third image features from the aortic wall and the intraluminal thrombus, the third image features being indicative of the calcification, said determining the presence of the calcification being performed by the second deep learning model using the third image features.

In one embodiment, the third deep learning model comprises a fully convolutional network (FCN)-based model.

In one embodiment, the third deep learning model comprises dilated convolutional layers.

In one embodiment, the third deep learning model comprises a binary classifier.

In one embodiment, the second deep learning model comprises a convolutional neural network (CNN).

In one embodiment, the image comprises a cross-sectional view of the body of the given subject.

In one embodiment, the image comprises a computational tomography slice.

According to a second broad aspect, there is provided a system for segmenting aortic tissues in images of a body of a given subject, the system comprising: a processor; a non-transitory storage medium operatively connected to the processor, the non-transitory storage medium comprising computer-readable instructions; the processor having access to at least one deep learning model having been trained to segment tissues in images, the processor, upon executing the computer-readable instructions, being configured for: receiving the image of the body of the given subject, the image comprising an aorta, an intraluminal thrombus and additional body parts; extracting a region of interest from the received image, the region of interest comprising the aorta and the intraluminal thrombus; determining, within the region of interest, a presence of a calcification on at least one of an aortic wall and the intraluminal thrombus; and outputting an indication of the presence of the calcification.

In one embodiment, the at least one deep learning model comprises a first deep learning and a second deep learning model, said extracting the region of interest being performed by the first deep learning model and said determining the presence of the calcification is performed by the second deep learning model.

In one embodiment, the step of extracting the region of interest comprises: extracting, using the first deep learning model, first image features from the image, the first image features being indicative of the aorta and the intraluminal thrombus; and segmenting, using the first deep learning model, the region of interest from the image using the first image features.

In one embodiment, the first deep learning model comprises a fully convolutional network (FCN)-based model.

In one embodiment, the first deep learning model comprises dilated convolutional layers.

In one embodiment, the first deep learning model comprises a binary classifier.

In one embodiment, the processor is further configured for detecting an aortic lumen of the aorta within the region of interest, the aortic lumen, the aortic wall and the intraluminal thrombus forming together the region of interest.

In one embodiment, the image further comprises at least one artery, the at least one artery being part of the region of interest, and said detecting the aortic lumen further comprises detecting the artery lumen of the at least one artery.

In one embodiment, the at least one artery comprises at least one of: at least one common iliac artery, at least one internal iliac artery and at least one external iliac artery.

In one embodiment, the step of detecting the aortic lumen is performed by the second deep learning model, said detecting the aortic lumen and said determining the presence of the calcification being concurrently performed by the second deep learning model.

In one embodiment, the second deep learning model is configured for: extracting second image features from the region of interest, the second image features being indicative of the aortic lumen and the calcification, said determining the presence of the calcification being performed using the second image features.

In one embodiment, the second deep learning model comprises a ResNet-based fully conventional network model trained for multi-class segmentation.

In one embodiment, the at least one deep learning model further comprises a third deep learning model, said detecting the aortic lumen being performed by the third deep learning model, the method further comprising removing the aortic lumen from the region of interest, thereby obtaining the aortic wall and the intraluminal thrombus.

In one embodiment, the step of detecting the aortic lumen comprises: extracting, using the third deep learning model, second image features from the region of interest, the second image features being indicative of the aortic lumen; and segmenting, using the third deep learning model, the aortic lumen from the region of interest using the second image features; and wherein the second deep learning model is configured for: extracting third image features from the aortic wall and the intraluminal thrombus, the third image features being indicative of the calcification, said determining the presence of the calcification being performed by the second deep learning model using the third image features.

In one embodiment, the third deep learning model comprises a fully convolutional network (FCN)-based model.

In one embodiment, the third deep learning model comprises dilated convolutional layers.

In one embodiment, the third deep learning model comprises a binary classifier.

In one embodiment, the second deep learning model comprises a convolutional neural network (CNN).

In one embodiment, the image comprises a cross-sectional view of the body of the given subject.

In one embodiment, the image comprises a computational tomography slice.

According to a further broad aspect, there is provided a non-volatile memory having stored thereon statements and instructions that upon execution by a processor perform the steps of the above-described method.

### Definitions

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression “a server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “server” and “third server” is not intended to imply any particular order, type, chronology, hierarchy or ranking (for example) of/between the servers, nor is their use (by itself) intended to imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 illustrates a schematic diagram of an electronic device, in accordance with one or more non-limiting embodiments of the present technology.

FIG. 2 illustrates a schematic diagram of a system, in accordance with one or more non-limiting embodiments of the present technology.

FIG. 3 illustrates a schematic diagram of an aortic tissue segmentation procedure executed using a set of three deep learning (DL) models, in accordance with one or more non-limiting embodiments of the present technology.

FIG. 4 illustrates a schematic diagram of an aortic tissue segmentation procedure executed using a set of two DL models, in accordance with one or more non-limiting embodiments of the present technology.

FIG. 5 illustrates a schematic diagram of a CT image input into a first DL model implemented as a ResNet-based encoder-decoder, a second DL model implemented as a ResNet-based encoder-decoder and a third DL model implemented as a ResNet-based CNN with classification layers, in accordance with one or more non-limiting embodiments of the present technology.

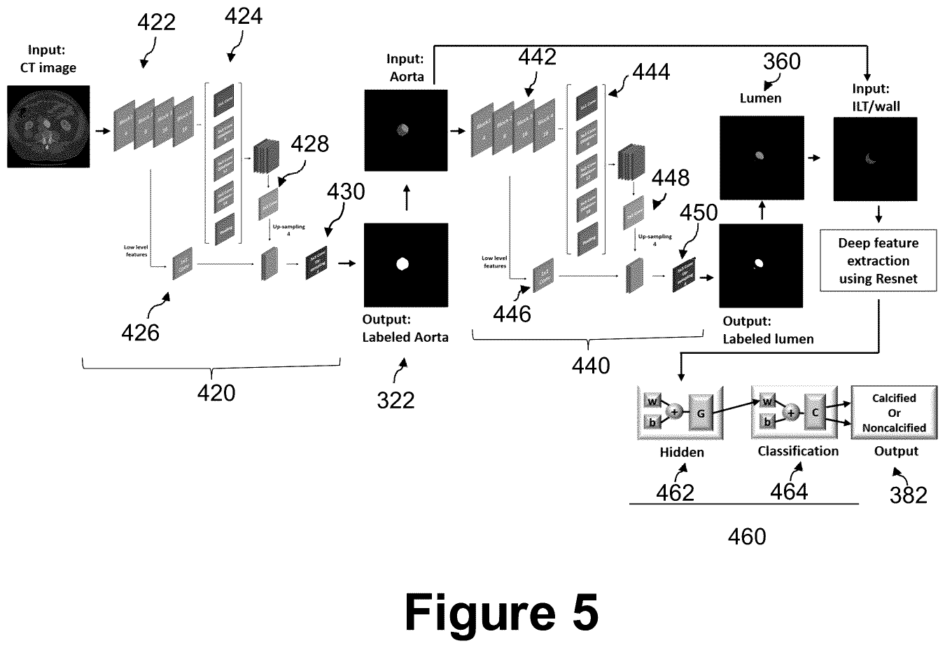

FIG. 6 illustrates a schematic diagram of a CT image, a region of interest (ROI) comprising an aorta and segmented tissues thereof output by using the set of DL models, in accordance with one or more non-limiting embodiments of the present technology.

FIG. 7 illustrates a plot of patient ID (x-axis) and the model performance or model accuracy (y-axis) having been generated by using leave-one-out cross-validation, in accordance with one or more non-limiting embodiments of the present technology.

FIG. 8 illustrates examples of images comprising an aorta, annotated ground-truths, extracted lumens and the extracted aorta, in accordance with one or more non-limiting embodiments of the present technology.

FIG. 9 illustrates examples of images resulting from segmentation for four different patients data including extracted aorta (the output of the first step), annotated ground-truth, lumen detection, extracted lumen and remaining tissues including wall and thrombus, the examples being illustrated, in accordance with one or more non-limiting embodiments of the present technology.

FIG. 10 illustrates examples of plots of network performance vs optimal number of epochs, hidden size, Sigma, and network validation failures respectively obtained by evaluating the performance of the third DL model, in accordance with one or more non-limiting embodiments of the present technology.

FIG. 11 illustrates a flow chart of a method for segmenting aortic tissues in images, in accordance with one or more non-limiting embodiments of the present technology.

FIG. 12 illustrates a particular implementation of the method of FIG. 11 in which three DL models are used, in accordance with one or more non-limiting embodiments of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting embodiments of the present technology, the processor may be a general-purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting examples to illustrate various implementations of aspects of the present technology.

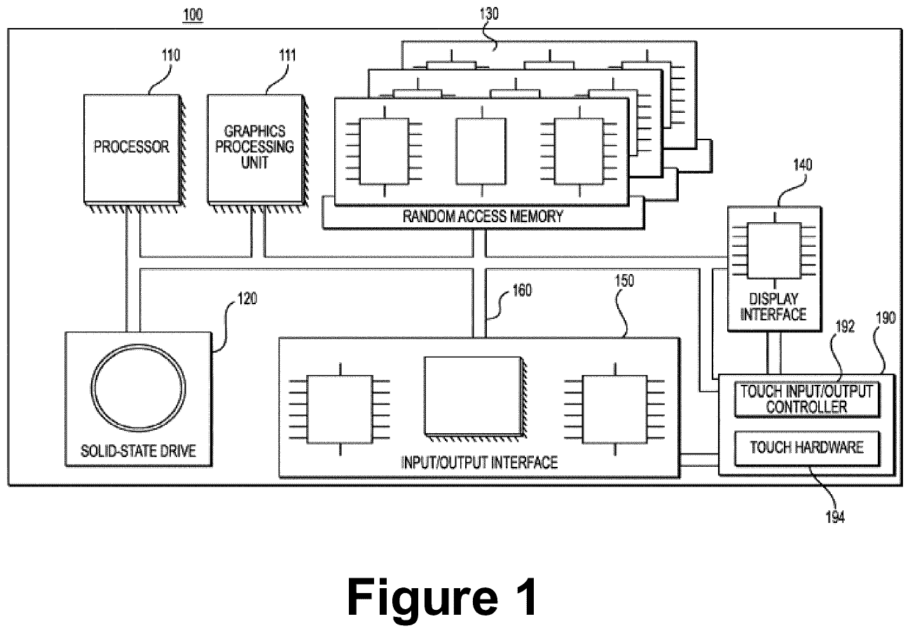

With reference to FIG. 1, there is illustrated a schematic diagram of an electronic device 100 suitable for use with some non-limiting embodiments of the present technology.

Electronic Device

The electronic device 100 comprises various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the electronic device 100 may be enabled by one or more internal and/or external buses 160 (e.g. a PCI bus, universal serial bus, IEEE 1394 “Firewire” bus, SCSI bus, Serial-ATA bus, etc.), to which the various hardware components are electronically coupled.

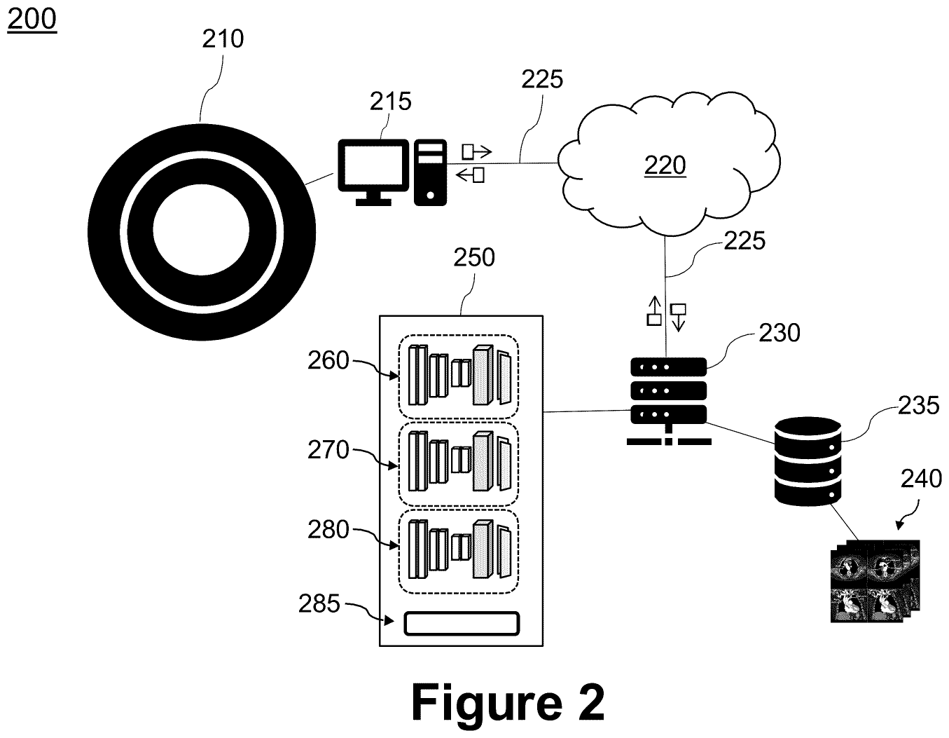

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 2, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the electronic device 100 in addition or in replacement of the touchscreen 190.

According to implementations of the present technology, the solid-state drive 120 stores program instructions suitable for being loaded into the random-access memory 130 and executed by the processor 110 and/or the GPU 111 for segmenting aortic tissues using one or more machine learning models. For example, the program instructions may be part of a library or an application.

The electronic device 100 may be implemented in the form of a server, a desktop computer, a laptop computer, a tablet, a smartphone, a personal digital assistant or any device that may be configured to implement the present technology, as it may be understood by a person skilled in the art.

System

Referring to FIG. 2, there is shown a schematic diagram of a communication system 200, which will be referred to as the system 200, the system 200 being suitable for implementing non-limiting embodiments of the present technology. It is to be expressly understood that the system 200 as illustrated is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The system 200 comprises inter alia a medical imaging apparatus 210 associated with a workstation computer 215, and a server 230 coupled over a communications network 220 via respective communication links 225 (not separately numbered).

Medical Device

The medical imaging apparatus 210 is configured to inter alia: (i) acquire one or more images comprising at least a portion of an aorta of a given subject comprising an aneurysm. It should be understood that the acquired image illustrates a cross-section of the aorta.

The medical imaging apparatus 210 may comprise one of: a computed tomography (CT) scanner, a magnetic resonance imaging (MRI) scanner, a 3D ultrasound and the like.

In some embodiments of the present technology, the medical imaging apparatus 210 may comprise a plurality of medical imaging apparatuses, such as one or more of a computational tomography (CT) scanner, a magnetic resonance imaging (MRI) scanner, a 3D ultrasound, and the like.

The medical imaging apparatus 210 may be configured with specific acquisition parameters for acquiring images of the patient comprising at least a portion of the aorta of the patient.

As a non-limiting example, in one or more embodiments where the medical imaging apparatus 210 is implemented as a CT scanner, a CT protocol comprising pre-operative retrospectively gated multidetector CT (MDCT—64-row multi-slice CT scanner) with variable dose radiation to capture the R-R interval may be used.

As another non-limiting example, in one or more embodiments where the medical imaging procedure comprises a MRI scanner, the MR protocol can comprise steady state T2 weighted fast field echo (TE=2.6 ms, TR=5.2 ms, flip angle 110 degree, fat suppression (SPIR), echo time 50 ms, maximum 25 heart phases, matrix 256×256, acquisition voxel MPS (measurement, phase and slice encoding directions) 1.56/1.56/3.00 mm and reconstruction voxel MPS 0.78/0.78/1.5), or similar cine acquisition of the portion of aorta under study, axial slices.

The medical imaging apparatus 210 includes or is connected to a workstation computer 215 for inter alia data transmission.

Workstation Computer

The workstation computer 215 is configured to inter alia: (i) control parameters of the medical imaging apparatus 210 and cause acquisition of images; and (ii) receive and process the plurality of images from the medical imaging apparatus 210.

In one or more embodiments. the workstation computer 215 may receive images in raw format and perform a tomographic reconstruction using known algorithms and software.

The implementation of the workstation computer 215 is known in the art. The workstation computer 215 may be implemented as the electronic device 100 or comprise components thereof, such as the processor 110, the graphics processing unit (GPU) 111, the solid-state drive 120, the random-access memory 130, the display interface 140, and the input/output interface 150.

In one or more other embodiments, the workstation computer 215 may be integrated at least in part into the medical imaging apparatus 210.

In one or more embodiments, the workstation computer 215 is configured according to the Digital Imaging and Communications in Medicine (DICOM) standard for communication and management of medical imaging information and related data.

In one or more embodiments, the workstation computer 215 may store the images in a local database (not illustrated).

The workstation computer 215 is connected to a server 230 over the communications network 220 via a respective communication link 225. In one or more embodiments, the workstation computer 215 may transmit the images and/or multiphase stack to the server 230 and the database 235 for storage and processing thereof

Server

The server 230 is configured to inter alia: (i) receive an input or initial image having been acquired by the medical imaging apparatus 210, the image comprising the aorta of a subject and other body parts; (ii) access a set of at least one deep learning (DL) model 250 and optionally the subtraction unit 285; (iii) train the set of DL models 250 to perform segmentations of aortic tissues; (iv) use the set of DL models 250 to perform segmentations of tissues in order to identify calcifications within the input image.

While in the illustrated embodiment, the set of DL models 250 comprises three DL models 260, 270 and 280 and a subtraction unit 285, it should be understood that the number of DL models may vary as along as the set of DL models 250 contains at least one DL model and that the subtraction unit 285 may be omitted. For example, the set of DL models may comprise two DL models and no subtraction unit 285. In another example such as in the illustrated embodiment, the set of DL models may comprise three DL models and the subtraction unit 285.

How the server 230 is configured to do so is explained in more detail herein below.

The server 230 can be implemented as a conventional computer server and may comprise some or all of the components of the electronic device 100 illustrated in FIG. 2. In an example of one or more embodiments of the present technology, the server 230 can be implemented as a Dell™ PowerEdge™ Server running the Microsoft™ Windows Server™ operating system. Needless to say, the server 230 can be implemented in any other suitable hardware and/or software and/or firmware or a combination thereof. In the illustrated non-limiting embodiment of present technology, the server 230 is a single server. In alternative non-limiting embodiments of the present technology, the functionality of the server 230 may be distributed and may be implemented via multiple servers (not illustrated).

The implementation of the server 230 is well known to the person skilled in the art of the present technology. However, briefly speaking, the server 230 comprises a communication interface (not illustrated) structured and configured to communicate with various entities (such as the workstation computer 215, for example and other devices potentially coupled to the network 220) via the communications network 220. The server 230 further comprises at least one computer processor (e.g., a processor 110 or GPU 111 of the electronic device 100) operationally connected with the communication interface and structured and configured to execute various processes to be described herein.

In one or more embodiments, the server 230 may be implemented as the electronic device 100 or comprise components thereof, such as the processor 110, the graphics processing unit (GPU) 111, the solid-state drive 120, the random-access memory 130, the display interface 140, and the input/output interface 150.

The server 230 has access to the set of DL models 250.

Deep Learning (DL) Models

In the illustrated embodiment, the set of DL models 250 comprises a first DL model 260, a second DL model 270, and a third DL model 280. Each of the first DL model 260, the second DL model 270, and the third DL model 280 has been respectively trained to perform semantic segmentation of images, i.e., to classify an object class for each pixel within an image.

In one or more alternative embodiments, at least two of the first DL model 260, the second DL model 270 and the third DL model 280 may be implemented as a single DL model, as described in greater detail below.

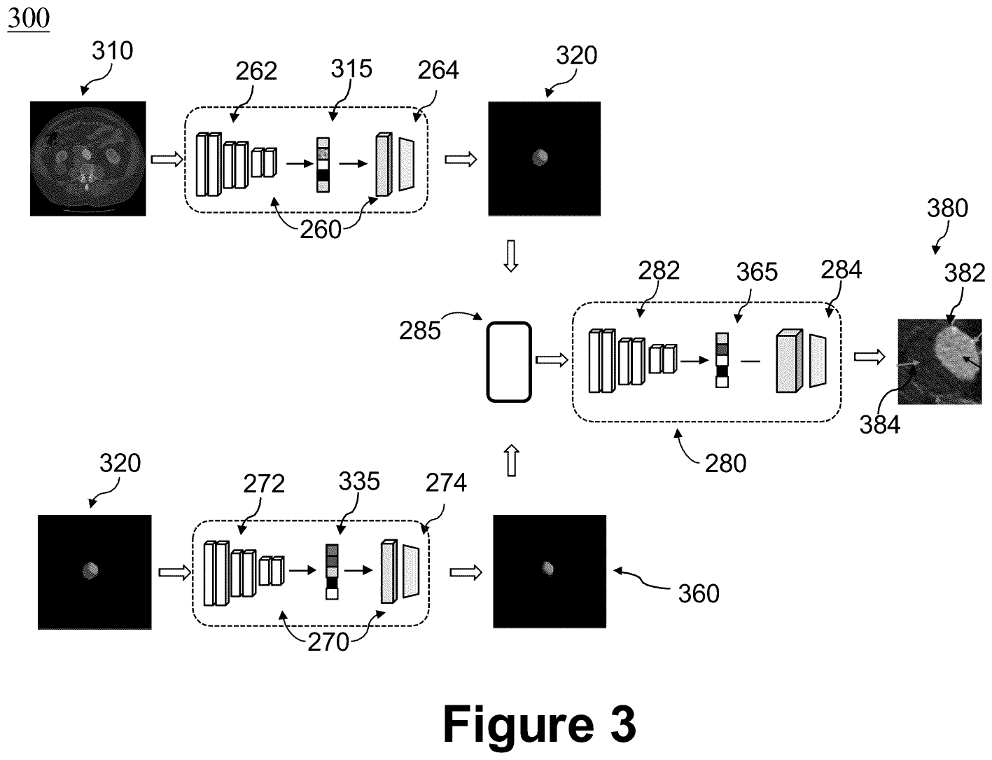

Each of the first DL model 260, the second DL model 270, and the third DL model 280 comprises a respective feature extractor 262, 272, 282 (shown in FIG. 3), and a respective prediction network 264, 274, 284 (shown in FIG. 3).

The first DL model 260 is configured to inter alley (i) receive the input or initial image; (ii) extract, via the respective feature extractor 262, a first set of image features therefrom; and (iii) segment, via the respective prediction network 264, based on the first set of image features, a region of interest (ROI) and a background in the input image in order to output the ROI. The ROI corresponds to the foreground of the input image, i.e., it corresponds to the input image from which the background has been removed.

The input image comprises an aorta, an ILT and other body parts such as organs and tissues to be removed for the further analysis. The ROI then comprises the aorta (including the aortic wall and the aortic lumen) and the ILT. In one embodiment, the input image comprises the aorta, the ILT and the common iliac arteries, the external iliac arteries and/or the external iliac arteries, in addition to the other body parts. In this case, the common iliac arteries, the external iliac arteries and/or the external iliac arteries are contained into the ROI.

In one embodiment, the other body parts to be removed from the input image comprise the spine, the kidneys, the mesenteric artery of the subject, and/or the like.

The first DL model 260 is trained to perform semantic segmentation of images. The first DL model 260 is configured to perform foreground and background segmentation, i.e. binary segmentation. In this case, the foreground comprises the aorta and the ILT, and optionally the common iliac arteries, the external iliac arteries and/or the external iliac arteries, if present in the input image, and the background comprises the remaining of the input image, i.e. the other body parts described above.

In one or more other embodiments, the first DL model 260 may be trained to perform multi-class semantic segmentation.

In one or more embodiments, the first DL model 260 has an encoder-decoder architecture.

In one or more embodiments, the first DL model 260 is implemented as a fully convolution network (FCN) such as a residual network (ResNet)-based FCN.

In a residual network (ResNet), building blocks are stacked on top of each other and each of them is a combination of convolutional layers with kernel sizes of 1×1, 3×3, and 5×5. The output filter banks from each building block are concatenated into a single output vector that is used as the input for the next stage. 1×1 convolutions are used for dimensionality reduction. The first DL model 260 uses dilated convolutions which are parametrized by a dilation rate assigned to the convolutional layer(s). Dilated convolutions, by maintaining the same stride, number of parameters, and computational cost, enable the kernel to take into account a larger filed of view at each convolutional layer, in contrast with standard patch-based CNNs. The use of dilated convolutions results in denser output feature and higher segmentation performance compared to networks with standard convolutional layers. Dilated convolutions are applied by using equation (1):)

y[i]=Σₖx[i+r,k]w(k) (1)

where i is a location in output y. The dilated convolution with dilation rate i is applied over the feature map x with kernel w.

Thus, in some embodiments of the present technology, a ResNet-based FCN architecture enables accessing strong discriminating deep features and overcoming limitations of patch-based CNNs for segmentation tasks.

Non-limiting examples of ResNet include ResNet50 (50 layers), ResNet101 (101 layers), ResNet152 (152 layers), ResNet50V2 (50 layers with batch normalization), ResNet101V2 (101 layers with batch normalization), and ResNet152V2 (152 layers with batch normalization).

In one or more alternative embodiments, the first DL model 260 may be implemented based on one of: AlexNet, GoogleNet, and VGG.

The second DL model 270 is configured to inter alia: (i) receive the region of interest (ROI) comprising at least the aorta and the ILT; (ii) extract, via the respective feature extractor 272, a second set of image features therefrom; and (iii) segment, via the respective prediction network 274 and based on the second set of image features 335, the ROI to obtain at least a segmented aortic lumen.

In an embodiment in which the ROI comprises arteries other than the aorta such as the common iliac arteries, the external iliac arteries and/or the external iliac arteries, the second DL model 270 is configured for segmenting the lumens of all arteries present in the ROI, e.g., the lumen of the aorta and the lumens of the common iliac arteries, the external iliac arteries and/or the external iliac arteries.

The second DL model 270 is trained to perform semantic segmentation of lumens in aortas. The second DL model 270 is configured to perform foreground and background segmentation, i.e. binary segmentation. In one or more other embodiments, the second DL model 270 may be trained to perform multi-class semantic segmentation.

Similarly to the first DL model 260, the second DL model 270 may be implemented as an FCN such as ResNet based FCN.

The subtraction unit 285 is configured to inter alia: (i) receive the ROI from the first DL model 260 and the lumen of the aorta from the second DL model 270; (ii) remove the identified lumen from the ROI; and (iii) output the ROI form which the lumen has been removed. The lumen present in the ROI may be seen as the foreground and the remaining of the ROI may be seen as the background of the ROI. In this case, the subtraction unit 285 may be seen as being configured to extract the background of the ROI from the ROI.

In an embodiment in which the only artery comprised in the ROI is the aorta, the subtraction unit 285 is configured for extracting or removing the lumen of the aorta from the ROI to output the background of the ROI which corresponds to the aorta walls and the ILT, i.e., the subtraction unit 285 outputs an image of the aorta wall and the ILT only.

In an embodiment in which the ROI comprises the aorta artery and at least another artery, such as at least one common iliac artery, at least one internal iliac artery and/or at least one external iliac artery, the subtraction unit 285 is configured for extracting or removing the lumen of each artery from the ROI to output the background of the ROI which corresponds to the walls of all the arteries and the ILT, i.e., the subtraction unit 285 outputs an image of the aorta wall, the ILT and the wall of any other artery contained in the ROI such as the wall of at least one common iliac artery, at least one internal iliac artery and/or at least one external iliac artery.

The third DL model 280 is configured to inter alia: (i) receive the background of the ROI from the subtraction unit 285, i.e., the ROI from which the lumen of any artery has been removed; (ii) extract, via the respective feature extractor 282, a third set of image features therefrom; and (iii) classify, via the respective prediction network 284 and based on the third set of image features, the input image as containing or not calcification.

The third DL model 280 is configured for identifying calcified tissue within the background of the ROI, i.e., within the ROI from which the lumen of any artery has been removed. In one or more embodiments, the third DL model 280 is configured for identifying calcification on the aortic wall and/or the ILT. In some other embodiments, the third DL model 280 is configured for identifying calcification on the aortic wall and/or the ILT and/or the wall of any artery other than the aorta, such on the wall of a common iliac artery, an internal iliac artery and/or an external iliac artery.

In one embodiment, the third DL model 280 is configured for classifying each pixel of the background of the ROI received from the subtraction unit 285 as being a pixel belonging to a calcified tissue or a pixel belonging to a non-calcified tissue.

In one embodiment, when the third DL model 280 identifies calcified tissue in the background of the ROI, the output of the third DL model 280 is an indication that the presence of calcification within the input image has been detected. It should be understood that any adequate indication may be used. For example, the indication may be a written indication, an audio indication, a visual indication, etc. In one embodiment, the indication comprises the background of the ROI received by the third DL model 280 in which the calcified tissue has been identified.

In one embodiment, when it identifies no calcification in the background of the ROI, the third DL model 280 outputs no indication. In another embodiment, the third DL model 280 may output an indication that no calcification has been detected within the input image.

In one or more embodiments, the third DL model 280 is implemented as a combination of a convolutional neural network (CNN) and a neural network. In one or more embodiments, the third DL model 280 comprises a CNN as a feature extractor and a feed forward neural network as a classifier.

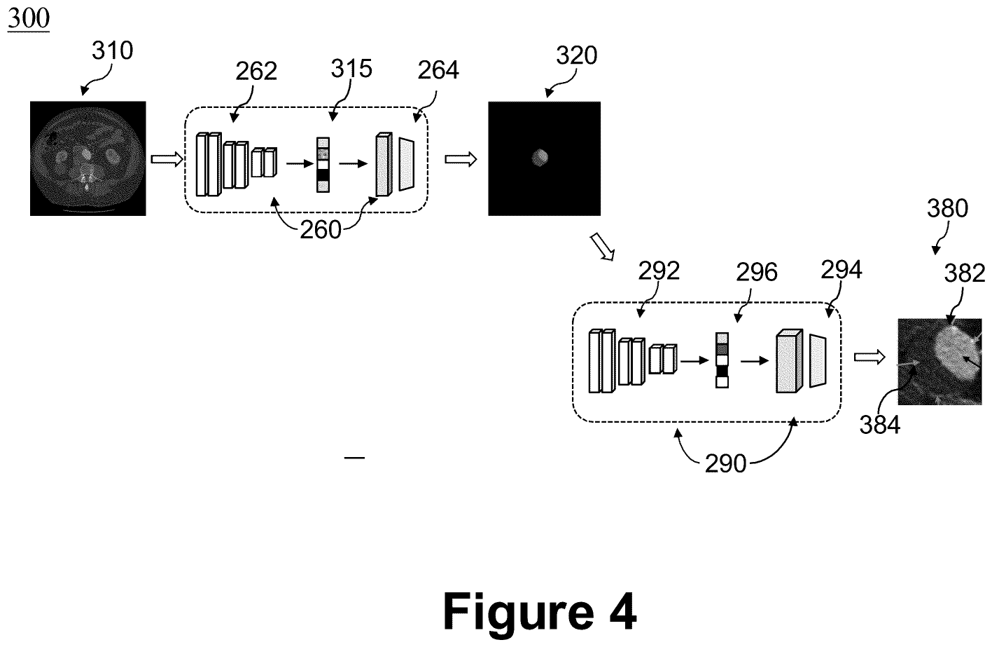

In another embodiment, the set of DL models 250 comprises two DL models, i.e., a first DL model and a second DL model, as illustrated in FIG. 4.

The first DL model is identical to the DL model 260, i.e., the first DL model is configured is configured to inter alia: (i) receive the input or initial image; (ii) extract, via the respective feature extractor 262, a first set of image features therefrom; and (iii) segment, via the respective prediction network 264, based on the first set of image features, a region of interest (ROI) and a background in the input image in order to output the ROI.

The second DL model 290 is configured to inter alia: (i) receive the ROI segmented by the first DL model 260; (ii) extract, via a respective feature extractor 292, a second set of image features 296 therefrom; and (iii) classify, via a respective prediction network 294 and based on the second set of image features 296, the initial image as containing or not calcification. In one or more embodiments, the second DL model 290 is configured for identifying calcification on the aortic wall and/or the ILT.

In one embodiment, the second DL model 290 is configured to concurrently identify the lumen of any artery included in the ROI (including the lumen of the aorta) to detect the wall of any artery and the ILT, and detect any calcification on the wall of any artery and/or the ILT.

In one embodiment, the second DL model 290 is configured for multi-class segmentation to segment lumen and calcification in the ROI. In this case, the second DL model 290 is configured for identifying calcification within the ROI except in the region of the ROI identified as being the aortic lumen. In one embodiment, the second DL model 290 is a ResNet-based FCN model trained for multi-class segmentation.

In one embodiment, the system comprising the two DL models 260 and 290 allows for a better identification of the location of the calcification than the system comprising the three DL models 260, 270 and 280.

Database

The database 235 is configured to inter alia: (i) store medical images; (ii) store model parameters and hyperparameters for the set of DL models 250; (iii) store datasets for training, testing and validating the set of DL models 250; (iv) store segmentation output by the set of DL models 250.

The labelled training dataset 240 or set of labelled training examples 240 comprises a plurality of training examples, where each labelled training example is associated with a respective label. The labelled training dataset 240 is used to train the set of DL models 250 to perform segmentation of aortic tissues. Each image in the labelled training dataset 240 may be segmented with indications of aortas, arteries other than the aorta, lumens, ILT, calcification, if any.

It will be appreciated that the nature of the labelled training dataset 240 and the number of training data is not limited and depends on the task at hand. The training dataset 240 may comprise any kind of digital file which may be processed by a machine learning model as described herein to generate predictions.

In one or more embodiments, the database 235 may store DL file formats, such as .tfrecords, .csv, .npy, and .petastorm as well as the file formats used to store models, such as .pb and .pkl. The database 235 may also store well-known file formats such as, but not limited to image file formats (e.g., .png, .jpeg), video file formats (e.g., .mp4, .mkv, etc.), archive file formats (e.g., .zip, .gz, .tar, .bzip2), document file formats (e.g., .docx, .pdf, .txt) or web file formats (e.g., .html).

As a non-limiting example, the labelled training dataset 240 may comprise 6030 CT images obtained from 56 different patients affected by abdominal aortic aneurysm using a CT imaging apparatus (GE Medical Systems, Chicago, Illinois, U.S.) with data annotated by a trained operator using Simpleware™ (Synopsys Inc., Mountain View, California, U.S.) with manual annotations validated by clinicians.

It will be appreciated that the database 235 may store other types of data such as validation datasets (not illustrated), test datasets (not illustrated) and the like.

Communication Network

In some embodiments of the present technology, the communications network 220 is the Internet. In alternative non-limiting embodiments, the communication network 220 can be implemented as any suitable local area network (LAN), wide area network (WAN), a private communication network or the like. It should be expressly understood that implementations for the communication network 220 are for illustration purposes only. How a communication link 225 (not separately numbered) between the workstation computer 215 and/or the server 230 and/or another electronic device (not illustrated) and the communications network 220 is implemented will depend inter alia on how each of the medical imaging apparatus 210, the workstation computer 215, and the server 230 is implemented.

The communication network 220 may be used in order to transmit data packets amongst the workstation computer 215, the server 230 and the database 235. For example, the communication network 220 may be used to transmit requests between the workstation computer 215 and the server 230.

Aortic Tissue Segmentation Procedure

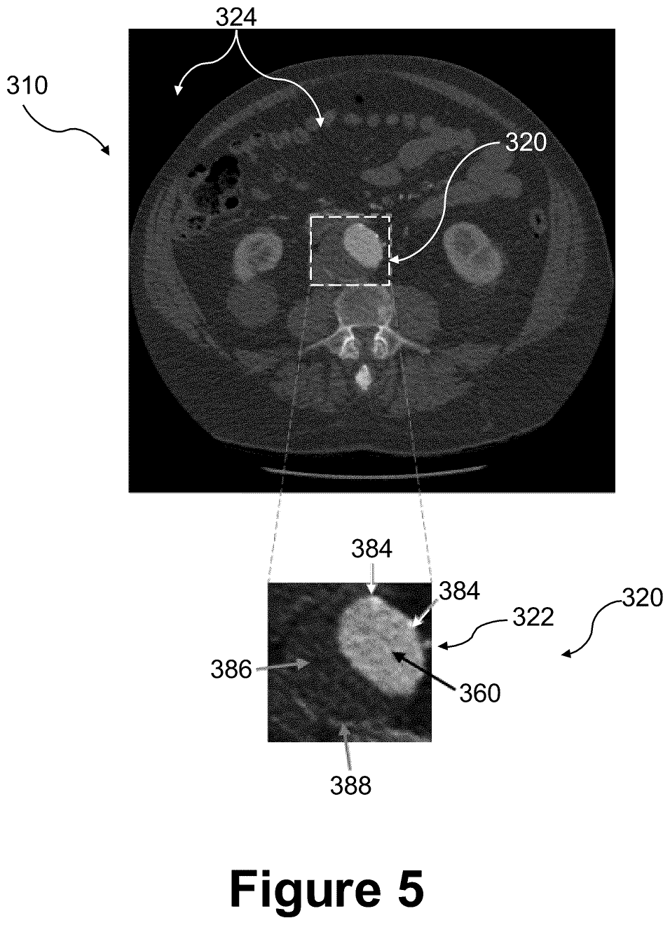

With reference to FIG. 3 and to FIG. 6, there is illustrated a schematic diagram of an aortic tissue segmentation procedure 300 and exemplary segmented tissues in the aorta obtained during the aortic tissue segmentation procedure 300 in accordance with one or more non-limiting embodiments of the present technology.

In one or more embodiments, the aortic tissue segmentation procedure 300 may be executed by the server 230. It is contemplated that some portions of the aortic tissue segmentation procedure 300 may be executed in parallel by the server 230 or by electronic devices (such as the workstation computer 215) as will be recognized by persons skilled in the art.

The aortic tissue segmentation procedure 300 uses the set of DL models 250 to perform semantic segmentation of aortic tissues and identify calcified tissue, if any. The set of DL models 250 have been pretrained to perform segmentation of aortic tissues during a training procedure and classification of calcified tissue and non-calcified tissue, which will be described in more detail herein below.

The aortic tissue segmentation procedure 300 obtains one or more initial or input images 310 (only one being depicted in FIG. 3) of a body of a given subject including at least the aorta, the ILT and other body parts to be removed for analysis purposes, as described above. The image may be received from the medical imaging apparatus 210, the database 235, or from any other electronic device (not illustrated) connected to the server 230.

In one or more embodiments, the image 310 is a cross-sectional view. The image may be a CT image of the aortic wall for example. It is contemplated that other imaging modalities such as MRI may be used without departing from the scope of the present technology.

As described above, the image 310 may include one or more organs of the given patient other than the aorta. As a non-limiting example, the image 310 may be a cardiac CT scan for example. In one or more embodiments, the image 310 has been acquired by the medical imaging apparatus 210 without using contrast mediums.

It will be appreciated that the image 310 may be preselected from a stack of images based on various criteria. In one or more other embodiments, the image 310 may not be preselected and may have been chosen randomly from a stack of CT images of the patient.

The first DL model 260 receives the image 310. More specifically, the respective feature extractor 262 of the first DL model 260 receives as an input the image 310.

The first DL model 260 extracts, via the respective feature extractor 264, a first set of image features 315 from the image 310. The first set of image features 315 comprises deep features indicative of the presence of the aorta and the ILT in the image 310. In one embodiment, the deep features are further indicative of the presence of at least another artery such as at least one common iliac artery, at least one external iliac artery and/or at least one internal iliac artery. In one embodiment, the deep features are a combination of abstract level features, such as shape and border, and detailed image information such as texture features.

In ne embodiment, the first set of image features 315 comprises deep features including one or more of textural, geometrical, topological and structural features indicative of the presence of the aorta and the ILT in the image 310, and optionally of the presence of at least another artery.

The respective prediction network 264 of the first DL model 260 uses the first set of image features 315 to obtain a region of interest (ROI) 320 and a background 324 of the image 310. The ROI 320 comprises at least the aorta 322 (including the aortic lumen 360 and the aortic wall 388), the ILT 386, and the calcification 384, if any, (and optionally at least another artery), and the background 324 comprises the remaining of the image 310 which comprises remaining body parts such as other organs and tissues (such as the spine), and other portions of the image 310 that are not needed for further analysis.

In one or more embodiments, the respective prediction network 264 segments the image 310, i.e. it classifies each pixel in the image 310 as belonging to the ROI 320 or belonging to the background 324.

The first DL model 260 outputs the ROI 320, i.e. the portion of the input image 310 that contains the aorta, the ILT and optionally at least another artery. In one or more embodiments, the aortic tissue segmentation procedure 300 may subtract the background 324 from the image 310 to obtain the ROI 320, and output the ROI 320.

It will be appreciated that the ROI 320 may only be a portion of the image 310.

In one or more embodiments, such as when the first DL model 260 has been provided with an image of the same subject acquired at a different moment in time, the first DL model 260 may further identify geometrical changes in the aorta and/or the ILT by using a network trained for this purpose (e.g. shallow network comprising fewer hidden layers).

In an embodiment in which the set of DL models 250 comprises three DL models 260, 270 and 280, the second DL model 270 receives as an input the ROI 320 identified by the first DL model 260.

The second DL model 270 extracts, via the respective feature extractor 272, a second set of image features 335 from the ROI 320.

The second set of image features 335 are indicative of the presence of lumens, such as the aortic lumen 360, in the ROI 320. In one embodiment, the second set of image features 335 comprise deep features which are a combination of abstract level features, such as shape and border, and detailed image information such as texture features.

It will be appreciated that the second set of image features 335 are generally different from the first set of image features 315. However it is contemplated that in alternative embodiments, at least a subset of the first set of image features 315 and at least a subset of the second set of image features 335 may be shared.

The second DL model 270 segments, via the respective prediction network 274 and based on the second set of image features 335, the ROI 320 to identify any artery lumen such as the aortic lumen 360 within the ROI 320 and the remaining of the ROI 320 (hereinafter referred to as the background of the ROI 360) comprises the aortic wall 388, the ILT 386, the calcification 384, if any, and optionally the wall of any other artery, such as the wall of at least one common iliac artery, at least one internal iliac artery and/or at least one external iliac artery. The second DL model 270 classifies each pixel in the ROI 320 as being one of a lumen 360 and a non-lumen (i.e., background). In one embodiment, the background of the ROI 320 comprises the aortic wall and the ILT. In an embodiment in which the input image 310 comprises the common iliac arteries, the internal iliac arteries and/or the external iliac arteries, the background of the ROI 320 further comprises the walls of the common iliac arteries, the internal iliac arteries and/or the external iliac arteries.

In one embodiment, the second DL model 270 outputs the lumen identified in the ROI 320. In one embodiment, the output of the second DL model 270 is an image of the identified lumen(s). In another embodiment, the output of the second DL model 270 is an identification of the pixels of the ROI 320 that have been identified as belonging to a lumen.

It will be appreciated that the performance of the second DL model 270 in obtaining the lumen 360 is improved due to the extraction and segmentation of the ROI 320 by the first DL model 260 beforehand.

The subtraction unit 285 receives as inputs the ROI 320 from the first DL model 260 and the lumen(s) from the second DL model 270, and removes the lumen(s) from the ROI 320 to obtain the background of the ROI 320.

The third DL model 280 receives as an input the background of the ROI 320 that contains the wall of the aorta, the ILT and optionally the wall of at least another artery, and identify calcified tissue, if any, in the background of the ROI 320. If calcified tissue is detected, the third DL model 280 outputs an indication that calcified tissue has been detected. The indication may be stored in memory along with the input image 310 so that the input image may be tagged as containing calcified tissue. In another example, the indication may be provided for display on a display unit.

In one embodiment, when it determines that the background of the ROI 320 contains no calcified tissue, the third DL model 280 outputs an indication that no calcified tissue has been detected in the input image 310. The indication may be stored in memory along with the input image 310 so that the input image may be tagged as containing no calcified tissue. In another example, the indication may be provided for display on a display unit.

More specifically, the third DL model 280 extracts, via the respective feature extractor 282, a third set of image features 335 from the background of the ROI 320.

The third set of image features 365 are indicative of the presence of calcified tissue in the background of the ROI 320. In one embodiment, the third set of image features 365 comprise deep features which are a combination of abstract level features, such as shape and border, and detailed image information such as texture features.

The third DL model 280 segments, via the respective prediction network 284 and based on the third set of image features 365, the background of the ROI 320 to identify any calcified tissue. The third DL model 280 classifies each pixel in the background of the ROI 320 as belonging to a calcified tissue or belonging to a non-calcified tissue

In one or more embodiments, the feature extractor 282 is implemented as a ResNet CNN.

In one or more embodiments, the prediction network 284 is implemented as a feed forward neural network.

With brief reference to FIG. 4, there is shown a non-limiting example of the first DL model 260 implemented as a ResNet-based encoder-decoder 420 with dilated convolutions, the second DL model 270 implemented as a ResNet-based encoder-decoder 440 with dilated convolutions and the third DL model 280 implemented as ResNet-based classifier 460.

The ResNet-based encoder-decoder 420 comprises a plurality of blocks 422 of size 4, 8, 16 and 16 and a plurality of layers 424 comprising a 1×1 convolution layer, a 3×3 dilated convolution layer, a 3×3 dilated convolution layer and a pooling layer.

The ResNet-based encoder-decoder 420 comprises another convolution layer 426 in the form of a 1×1 convolution layer for processing low-level features of the first set of image features output by the plurality of blocks 422, the another convolution layer 426 being stacked with a first up-sampling convolution layer 428 in the form of a 1×1 convolution layer 426 for up-sampling the first set of image features. The last up-sampling convolution layer 430 is in the form a 3×3 convolution layer that outputs the ROI 320.

Similarly, the ResNet-based encoder-decoder 440 comprises another convolution layer 446 in the form of a 1×1 convolution layer for processing low-level features of the second set of image features output by the plurality of blocks 442, the another convolution layer 446 being stacked with a first up-sampling convolution layer 448 in the form of a 1×1 convolution layer 436 for up-sampling the second set of image features. The last up-sampling convolution layer 450 is in the form a 3×3 convolution layer that outputs the segmented lumen(s).

The ResNet-based classifier 460 comprises hidden CNN layers 462 which receives the background of the ROI 320 (i.e. the ROI 320 from which the identified lumen(s) has(ve) been removed) and generates a third set of deep image features that are processed by the feed forward classification layers 464 to output an indication of a presence of calcifications 382 in the background of the ROI 320.

In an embodiment in which the set of DL models 250 comprises only two DL models, the first DL model may be the DL model 260 implemented as a ResNet-based encoder-decoder 420 with dilated convolutions. The second DL model may be a Fully Convolutional Residual Network (ResNet-based FCN) trained for multi-class segmentation to detect concurrently lumen(s) and calcification in the ROI 320.

In one embodiment, a system comprising only two DL models allows for an improved detection of the location of calcifications, and a more accurate segmentation of the lumen(s) since it reduces the risk of considering a calcification as a lumen.

Training Procedure

During training of the set of DL models 250, the deeper network layers are fined-tuned by using grid searching for an extensive interval of values. It will be appreciated that the upper layers in the network architecture of the set of DL models 250 extract more generic features of the images such as edge, borders, and shapes, which are common attributes in various applications. To keep the weights of the other layers constant, the learning rate is forced to zero. The optimal learning parameters may be obtained by evaluating the model performance on a validation dataset for each assigned value. In one or more embodiments, the optimal learning parameter was determined to be 0.02. The momentum and scheduling rate were assigned as 0.8 and at each step of fine-tuning. The dilation rate was assigned as 2 and 4 for the last two blocks. An up-sampling factor of 8 was assigned to the decoder to up-sample the encoder output for the third DL model 280. The output of the decoder of the third DL model 280 was combined with the low level features after applying a 1×1 convolution.

Since the aorta usually represents a small fraction of the whole image, a weighted loss function is used. The performance of the set of DL models 250 is evaluated using both weighted cross-entropy and weighted generalized dice as the loss function.

In one implementation, weighted cross-entropy demonstrated an overall better performance with the weight defined by using equation (2):

$$w = ( N - {\sum }_{n} {p}_{n} ) / {\sum }_{n} {p}_{n} \qquad ( 2 )$$

where N is the number of images annotated as foreground with predicted probabilistic map elements pₙ.

In one implementation, Adam is applied as the network optimizer with a L2 regularization of 0.0005, a mini batch size of 10, and a validation patience of 6. To train the set of DL models 250, 80% of the data may be used for training the set of DL models 250, the remaining 20% may be split in two for validation and test datasets.

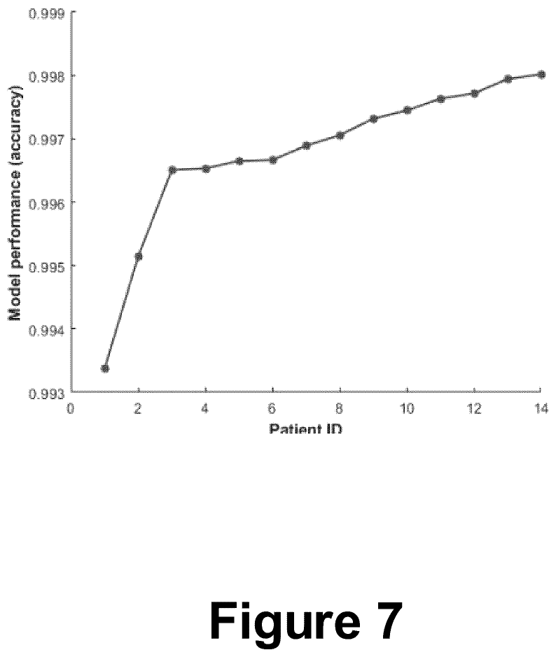

To validate the performance of the set of DL models 250 on new subject data, leave-one-out cross validation may be performed by leaving one subject data as the validation set and training by the set of DL models 250 on the data of all the other subjects. The validation process may be repeated for 32 times for a sub-set of 32 different subjects, and the results are shown in FIG. 7, with subject/patient ID (x-axis) and the model performance or model accuracy (y-axis).

To evaluate the results of the training of the set of DL models 250, at each step, the accuracy, sensitivity, specificity, and BF-score may be calculated.

In one or more embodiments, by using the confusion matrix, the per class Accuracy, Sensitivity, and Specificity and BF is calculated by using equations (3)-(7):

$$\begin{matrix}Accuracy = \frac{TP + TN}{TP + TN + FP + FN} & ( 3 ) \\ Sensitivity = \frac{TP}{TP + FN} & ( 4 ) \\ Specificity = \frac{TN}{TN + FP} & ( 5 ) \\ Precision = \frac{TP}{TP + FP} & ( 6 ) \\ BF score = \frac{2 \times Precision \times Sensitivity}{Precision + Sensitivity} & ( 7 )\end{matrix}$$

where TP, FP, FN, and TN are True Positive, False Positive, False Negative, and True Negative respectively

Table I shows results of obtained accuracy, sensitivity, specificity and BF for segmented tissues during training:

TABLE 1
Measured Accuracy, Sensitivity, Specificity, And Bf-Score For Extraction
Of Aorta Using Ct Images.

| Tissue type | Accuracy | Sensitivity | Specificity | BF-score |
|---|---|---|---|---|
| Aorta | 0.91 ± 0.09 | 0.90 ± 0.10 | 0.94 ± 0.05 | 0.91 ± 0.05 |
| Surrounding | 0.97 ± 0.03 | 0.99 ± 0.01 | 0.99 ± 0.01 | 1.00 ± 0.00 |
| tissues and | | | | |
| background | | | | |

Table 2 shows results of obtained accuracy, sensitivity, specificity and BF for extraction of the lumen in CT images of the aorta:

TABLE 2
Measured accuracy, sensitivity, specificity, and BF-Score for extraction
of the lumen in CT images of the aorta.

| Tissue type | Accuracy | Sensitivity | Specificity | BF-Score |
|---|---|---|---|---|
| Lumen | 0.99 ± 0.01 | 0.99 ± 0.01 | 0.99 ± 0.01 | 1.00 ± 0.00 |
| Surrounding | 0.99 ± 0.01 | 0.99 ± 0.01 | 0.99 ± 0.01 | 1.00 ± 0.00 |
| tissues and | | | | |
| background | | | | |

Table 3 shows results of obtained accuracy, sensitivity, specificity and BF for calcified ILT/wall versus non-calcified ILT/wall.

TABLE 1
Measured accuracy, sensitivity, and specificity for classification of
calcified ILT/wall versus non-calcified ILT/wall.

| Tissue type | Accuracy | Sensitivity | Specificity |
|---|---|---|---|
| Calcified | 0.91 | 0.91 | 0.90 |
| IL T/wall | | | |
| Non-calcified | 0.85 | 0.81 | 0.90 |
| IL T/wall | | | |

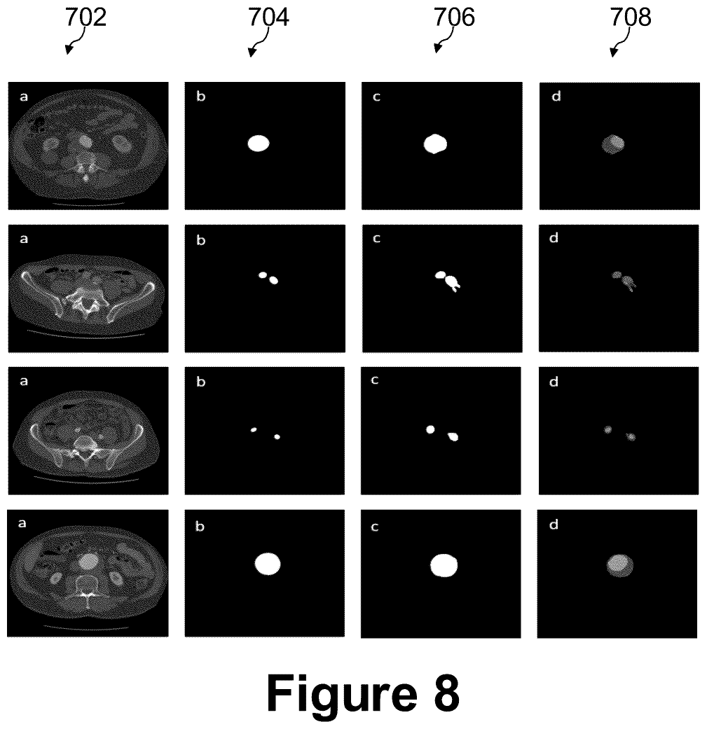

With reference to FIG. 8, there are illustrated exemplary images of CT-scans 702 (labelled with letter a) each comprising an aorta, images of annotated aortas used as ground truths 704 (labelled with letter b), model decisions comprising segmented lumens 706 (labelled with letter c), and extracted aortas 708 (labelled with letter d).

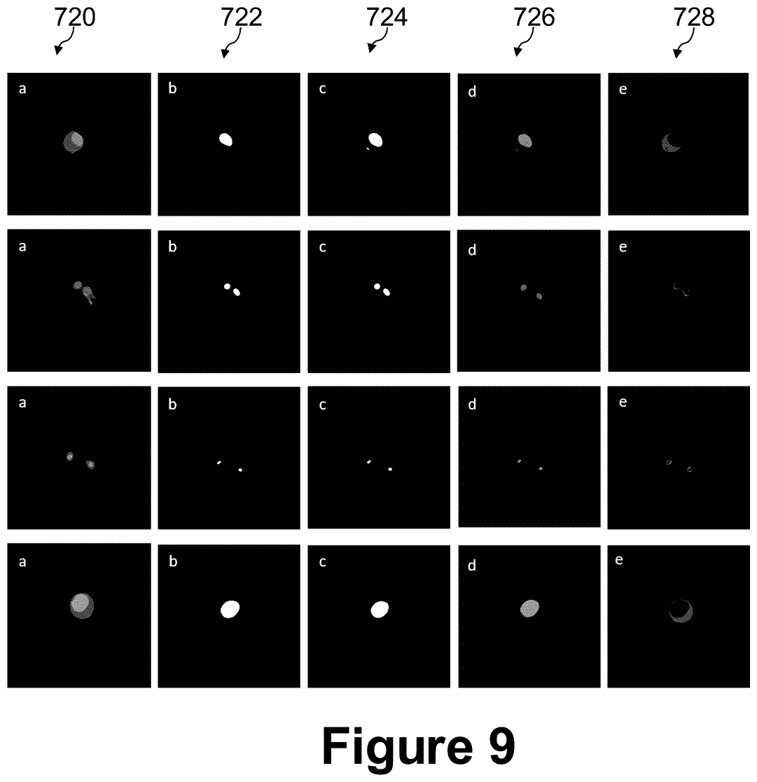

With reference to FIG. 9, there are illustrated exemplary images of results of segmentation for four different patient data: extracted aortas 720 (labelled with letter a) annotated ground-truths 722 (labelled with letter b), model decisions for lumen detection (labelled with letter c) extracted lumens 726 (labelled with letter d) and the artery wall and ILT 728 (labelled with letter e).

It should be noted that in accordance with one or more embodiments of the present technology, the aorta was extracted from the original image in the first step for two reasons. The first reason is the following. By considering the aorta as the ROI, the network is looking for extra features, which describes the relationship between lumen, thrombus, and wall instead of considering each tissue separately, which can reduce the error rate of the segmentation since all unwanted surrounding tissues are removed. Further, since the aorta is extracted from the whole image, only the lumen needs to be extracted from the aorta and the remaining tissues are a combination of ILT and artery wall (ILT/wall). Thus, the same configuration of a Resnet-based FCN may be adapted for lumen segmentation. The output of the previous step (aorta) is fed into the network for further processing to detect and extract the lumen. The second reason is to facilitate and pinpoint the fluid dynamic analysis of CT images obtained from patients with AAA. Therefore, the accurate extraction of the aortic lumen improves the process of fluid dynamic analysis since manual segmentation of aorta is replaced by a high precision automated procedure which not only accelerates the analytical process but also avoids error-prone manual segmentation.

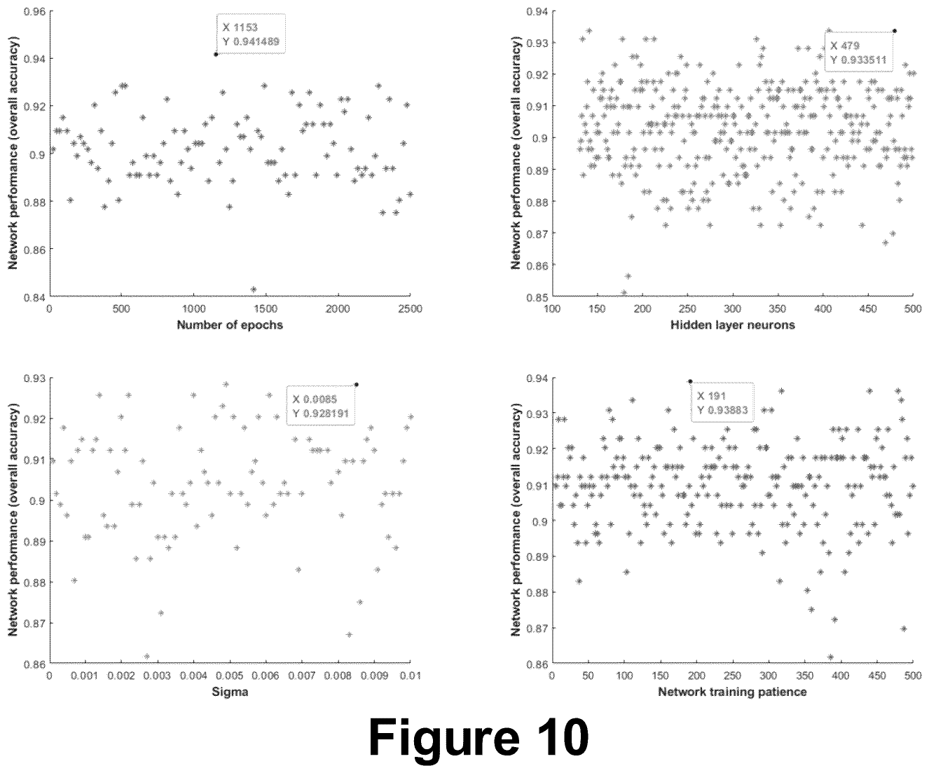

Finally, the thrombus and artery wall are evaluated to discriminate between calcified and non-calcified tissue. Since calcification may not occur in all cases, it may not always be efficient to train a FCN with a small number of images to detect calcification. With an existing dataset, it may be more efficient to discriminate between calcified and non-calcified ILT/wall by extracting deep features from all the images of the ILT/wall and train a classifier to consider the similarity between deep features and classify the calcified versus non-calcified images. To this end, in some embodiments, a combination of a CNN as feature extractor and a feed forward neural network as the classifier are used. All the ILT/wall images are labelled manually as calcified or non-calcified. To be consistent in using the networks and considering that ResNet is a strong CNN for feature extraction, features are extracted from all the images of ILT/wall obtained from the previous step. The extracted deep features are fed to a feed forward neural network with 479 hidden layer neurons, which acts as the classifier. To find the optimal hidden size for the network, the performance of the network is evaluated for an extensive interval of hidden size values from 100 to 500, as can be seen in FIG. 9. The training process is based on the scaled conjugate gradient method while the parameter Sigma is used to estimate the weight change for the second derivative approximation. To obtain the optimal value of Sigma, the performance of the classifier is evaluated by assigning various values from 0.0001 to 0.01 to Sigma. The highest performance of the network is obtained for the value 0.085, as can seen in FIG. 9. Training was performed for 1153 epochs with maximum validation failures of 191. For the number of epochs and validation failures, the performance of the network is evaluated for the values from 1 to 2500 and 0 to 500 respectively, as can be seen in FIG. 10.

Method Description

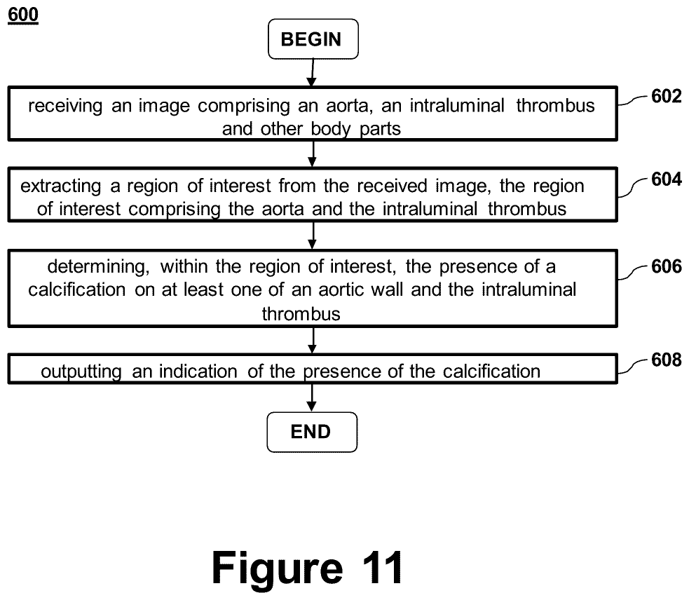

FIG. 11 illustrates a flowchart of a method 600 for segmenting aortic tissues to detect calcification in medical images, the method 600 being executed in accordance with one or more non-limiting embodiments of the present technology.

In one or more embodiments, the server 230 comprises a processing device such as the processor 110 and/or the GPU 111 operatively connected to a non-transitory computer readable storage medium such as the solid-state drive 120 and/or the random-access memory 130 storing computer-readable instructions. The processing device upon executing the computer-readable instructions, is configured to or operable to execute the method 800. It will be appreciated that the method 800 may be executed by more than one device.

The server 230 has access to the set of DL models 250, at least a portion of the set of DL models 250 having been trained to perform semantic segmentation of aortas in images acquired by a medical imaging apparatus. The server 230 may have also access to the subtraction unit 285. In one embodiment, the set of models 250 comprises the first DL model 260, the second DL model 270, and the third DL model 280. In another embodiment, the set of models 250 comprises the first DL model 260 and the DL model 290.

The method 600 begins at processing step 602.

At processing step 602, the processing device receives an image of a subject to be classified as containing or not a calcification on an aortic wall and/or an ILT. As described above, the image comprises an aorta, an ILT and other body parts such as organs and tissues to be removed for the further analysis. In one embodiment, the image comprises the aorta, the ILT and at least one artery such as at least one common iliac artery, at least one external iliac artery and/or at least one external iliac artery, in addition to the other body parts.

In one or more embodiments, the image 310 is a cross-sectional image comprising at least cross-sections of the aorta and the ILT. The image 310 may comprise CT-scan slice.

At processing step 604, the processing device extracts a ROI 320 from the image 310. The ROI 320 comprises the aorta and the ILT. In an embodiment in which the image 310 further comprises at least one artery such as at least one common iliac artery, at least one external iliac artery and/or at least one external iliac artery, the ROI 320 further comprises the at least one artery. It should be understood that at processing step 604, the above-mentioned other body parts are removed from the image 310 to obtain the ROI 320.

At processing step 606, the processing device determines whether the wall of the aorta 320 and/or the ILT contained in the ROI 320 contains calcified tissue or not.

If at processing step 606, it determines that the wall of the aorta and/or the ILT contains calcified tissue, then the processing device outputs an indication of the presence of the calcified tissue at processing step 608.

The method 600 then ends.

In one embodiment, the processing step 602 is performed by a first DL model such as the DL model 260.

In one embodiment, the method 600 further comprises a processing step of identifying at least the lumen of the aorta in ROI 320. In an embodiment in which the ROI 320 comprises at least one artery in addition to the aorta, the lumen of the artery is also identified.

In one embodiment, the identification of the lumen and the identification of the calcification are performed concurrently by a same DL model such as the DL model 290 which receives the ROI 320 as input.

In another embodiment, the identification of the lumen and the identification of the calcification are performed by different DL models. In this case, the identification of lumens within the ROI 320 is performed by a second DL model such the DL model 270, and the identification of the calcification is performed by a third DL model such as the DL model 280.

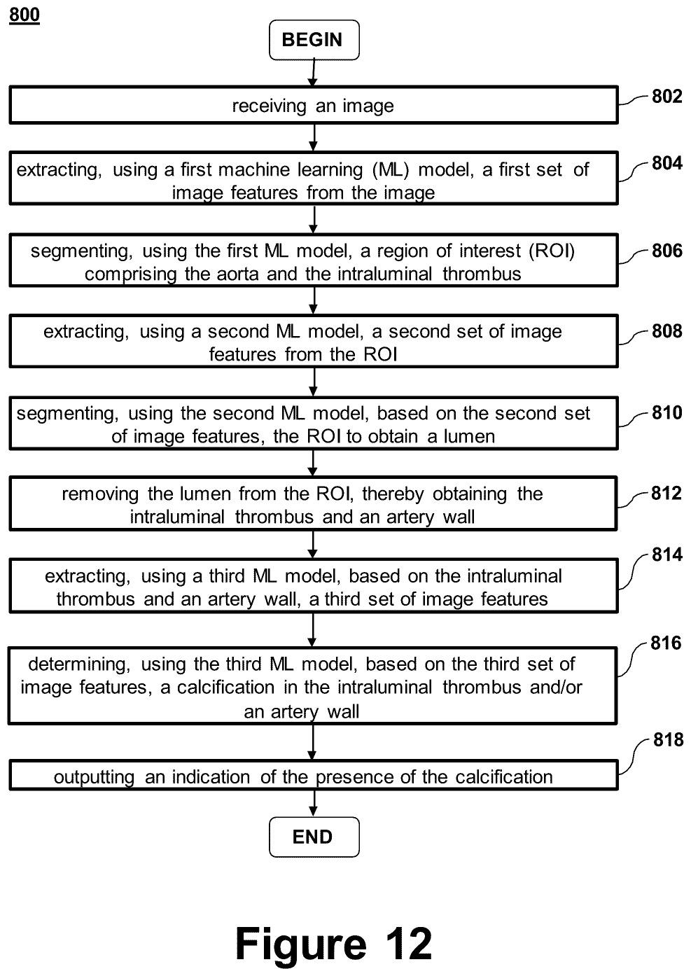

FIG. 12 illustrates a method 800 which corresponds to an exemplary embodiment of the method 600 in which the three DL models 260, 270 and 280 are used.

The method 800 starts at step 802.

At processing step 802, the processing device receives the image 310.

At processing step 804, the processing device uses the first DL model 260 to extract a first set of image features 315 from the image 310. In one or more embodiments, the processing device uses the respective feature extractor 264 of the first DL model 260 to extract the first set of image features 315 from the image 310. The first set of image features 315 are indicative of at least structural properties of an aorta in the image 310.

In one or more embodiments, the first DL model 260 is implemented as a ResNet-based encoder-decoder 420.

At processing step 806, the processing device uses the first DL model 260 to obtain the ROI 320 of the image 310 based on the first set of image features 315. In one or more embodiments, the processing device uses the respective prediction network 264 of the first DL model 260 to obtain ROI 320 of the image 310 based on the first set of image features 315.

In one or more embodiments, the first DL model 260 may comprise or be followed by a trained network (e.g. a trained neural network with one or two hidden layers) for identifying aortic geometrical variations based on the ROI 320 and a ROI acquired at a different moment in time.

At processing step 808, the processing device extracts, using the second DL model 270, a second set of image features 335 from the ROI 320. The processing device uses the respective feature extractor 272 of the second DL model 270 to extract and obtain the second set of image features 335 from the ROI 320. The second set of image features 335 are indicative of at least structural properties of lumen(s) present in the ROI 320.

In one or more embodiments, the second DL model 270 is implemented as a ResNet-based encoder-decoder similar to the first DL model 260.

At processing step 810, the processing device segments, based on the second set of image features 335, the ROI 320 to obtain at least the lumen 360 of the aorta 322 and optionally the lumen of any other artery included in the ROI 320. In one or more embodiments, the processing device segments the ROI 320 based on the second set of image features 335 by using the respective prediction network 274 of the second DL model 270 to obtain the lumen(s).

At processing step 812, the processing device removes the lumen(s) to identified in the ROI 320 from the ROI 320 to obtain the background of the ROI 320 which comprises the wall of the aorta and the ILT, and optionally the wall of any artery other than the aorta present in the ROI 320.

At processing step 814, the processing device extracts, by using the third DL model 280, a third set of image features 365 from the background of the ROI 320. In one or more embodiments, the third DL model 280 is implemented as a combination of a CNN and a neural network.

The processing device uses the respective feature extractor 282 of the third DL model 280 to extract the third set of image features 365. The third set of image features 365 are indicative of at least structural properties of calcifications.

At processing step 816, the processing device classifies, using the third DL model 280 based on the third set of image features 365, the tissues contained in the background of the ROI 320 as being calcified or non-calcified to obtain an indication of the presence of a calcification in the image 310. The processing device uses the respective prediction network 284 of the third DL model 280 to classify, based on the third set of image features 365, the tissues contained in the background of the ROI 320 as being calcified or non-calcified.

At processing step 818, the processing device outputs, when a calcification has been detected at step 816, an indication of the presence of the calcification in the image 310. When no calcification is detected at step 816, no indication may be outputted at step 818.

In one embodiment, the indication of the presence of the calcification comprises a segmented image with a visual indication of the calcification within the segmented image. As a non-limiting example the visual indication may include a colored contour and an indication of size of the calcification formation.

The method 800 then ends.

It will be appreciated that the method 800 does not require using preprocessing steps for processing the image 310, and that the set of DL models 250 may be trained on a relatively small dataset (e.g. 1000 images) as compared to standard segmentation techniques. Further, the method 800 does not require images having been acquired by using contrast mediums.

It should be expressly understood that not all technical effects mentioned herein need to be enjoyed in each and every embodiment of the present technology. For example, embodiments of the present technology may be implemented without the user enjoying some of these technical effects, while other non-limiting embodiments may be implemented with the user enjoying other technical effects or none at all.

Some of these steps and signal sending-receiving are well known in the art and, as such, have been omitted in certain portions of this description for the sake of simplicity. The signals can be sent-received using optical means (such as a fiber-optic connection), electronic means (such as using wired or wireless connection), and mechanical means (such as pressure-based, temperature based or any other suitable physical parameter based).

In the following, there is presented results of an experimental study.

### BACKGROUND

The present protocol summarizes the training and validation of the present segmentation model and addresses the inter-rater reliability by blind comparison of the results from two analysts for 19 retrospective patient's data.

Summary of Model Development & Training of Automatic Segmentation Model

The present segmentation model was designed considering the clinical needs of accurate and repeatable automated reconstruction of the aortic wall and structures of an aneurysm.

A Resnet-based FCN with dilated convolutions was employed for detection and extraction of the ROI including the aorta and iliac arteries. Fine-tuning was started from deeper network layers using grid searching for an extensive interval of values. Upper layers in the network architecture are responsible to extract more generic features of the images such as edge, borders, and shapes, which are common attributes in various applications. Caution was taken to allay over-fitting concerns in consideration of our patient data set. The weights of all other layers remained constant by forcing the learning rates to zero for those layers. The optimal learning parameters are obtained by evaluating the model performance on the validation set for each assigned value. The optimal learning parameter is determined to be 0.02. The momentum and scheduling rate is assigned as 0.8 and 0.9 at each step of the fine-tuning. Dilation rate is assigned as 2 and 4 for the last two blocks. An up-sampling factor of 8 is assigned to the decoder to up-sample the encoder output. The output of the decoder is combined with the low-level features after applying 1×1 convolution. The ROI is labeled as the first class and all the other surrounding tissues, and the image background is labeled as the second class. Since the ROI is a small fraction of the whole image, weighted loss functions are considered. The performance of the network is evaluated using both weighted cross-entropy and weighted generalized dice as loss functions. Weighted cross-entropy demonstrates better performance with the weight defined using equation (2).

Adam network optimizer is applied with L2 regularization of 0.0005, mini batch size of 8, and validation patience of 6.

The training is performed in three different steps:
1. Lumen, ILT, iliac arteries, and calcification: to train the model, 80% of the data is used for training, and the remaining 20% is split by two for validation and test sets.
2. Lumen/Calcification network: the same configuration of the network is employed for multi-class segmentation to detect lumen and calcification. In this case, three classes are defined as lumen, calcification, and background. Training is performed by assigning the 80% of the data to the training set, and the remaining 20% is split by two for validation and test sets.

In one embodiment, the DL model configuration is developed using a Resnet-based FCN with dilated convolution only for detection of the ROI and the lumen. The extracted aortic walls are analyzed using a combination of a CNN and a neural network to discriminate between calcified and non-calcified tissues. The training is performed using a first dataset including pre-operative dynamic CT images of patients selected for surgery (both open and EVAR), as well as three patients enrolled in the prospective study.

In one embodiment, the model is improved by retraining on more patient's data. The second dataset is part of a random selection of retrospective patients for which no information in terms of baseline or follow up is obtained. They were random patients with Abdominal Aortic Aneurysm CT imaging. For this dataset most of the cases has dynamic CT images, a few cases have static CT images.

In one embodiment, an improvement of the model is performed in two steps:
1. The second and third DP networks are replaced by a single multi-class segmentation network to concurrently detect both lumen and calcification in the ROI.
2. The patients are selected from a third dataset for retraining the automatic segmentation model. The third dataset was a selection of images from a retrospective study including patients that were diagnosed with Abdominal Aortic Aneurysm but not selected for surgery and were monitored with serial dynamic CT. For this dataset, information on which scan was a baseline and which was a follow up for each patient was available. The retraining was performed on the baselines.

The objective of the present scientific verification protocol is to validate the present segmentation model developed for characterizing various tissues in abdominal aortic aneurysm.

The verification of the segmentation model was performed on 19 baseline DICOM images selected from our third dataset. The patients have been never involved in training the model and they have never been analyzed by the model.

Methods

The present study is designed to verify the present segmentation method. For each patient, the inter-rater reliability is evaluated in three different steps: 1. The performance of the segmentation model is compared against the ground-truth. 2. The performance of the segmentation model is compared against a trained observer. 3. The segmentation created by the trained observer is compared against the ground-truth.

Test Procedure

For each DICOM CT slice, the original image was fed to the segmentation network. Pixel by pixel comparison was performed by calculating the confusion matrix between the output of the automatic segmentation and the corresponding ground-truth mask. The same calculation was performed between the output of automatic segmentation and the corresponding mask created by the trained observer. Using calculated confusion matrix for each slice, per-class accuracy, sensitivity, specificity, and BF-Score were measured. For all the 19 patients, the experiments were performed slice by slice and the results were saved for further statistical analysis.

Data Capture

The segmentation model has four different outputs (all in DICOM format): 1. Extracted aorta including a combination of the aortic wall, lumen, ILT, and calcification, 2. Extracted lumen, 3. Extracted calcification, 4. Detected landmark. All the calculated confusion matrices, accuracies, sensitivities, specificities, and BF-Scores were saved as .mat.

Data Analysis and Acceptance Criteria

The agreement between the automatic segmentation output, the corresponding ground-truth masks, and a separate trained observer are verified to identify individual bias.

The ground-truth was created by a trained observer which the quality and accuracy of the created masks were verified by an independent and experienced adjudicator. The ground-truth was created for all the 19 patients. The trained observer was blinded to the ground-truth to reduce the possibility of bias in the segmentation of the same DICOM images of 19 different patients.

For each patient, the automatic segmentation was applied to each slice. Then, the outputs of the segmentation networks were evaluated separately by calculating the confusion matrix for each output (ROI, lumen, and landmark). By defining the positive class as the ROI and negative class as the background and surrounding tissues, confusion matrix was calculated as follows:

| | |
|---|---|
| TP | FN |
| FP | TN |

Where TP, FP, TN and FN respectively stand for True Positive, False Positive, True Negative and False Negative.

TP represents the pixels that are correctly segmented as the ROI for each network, FP determines the background pixels that are incorrectly segmented as the ROI, TN represents the pixels that are correctly segmented as the background and surrounding tissues, and FN determines the ROI pixels that are incorrectly segmented as the background and surrounding tissues.

Using the confusion matrix, the general definitions of the accuracy, sensitivity, specificity, and BF-Score provided by equations (3) to (7) were used to measure per-class accuracy, sensitivity, specificity for full evaluation of the segmentation model in detecting and extracting the ROIs. The BF-Score evaluates how close the segmented boundary of the ROI matches the ground-truth boundary (see tables 4-6).

TABLE 4
Measured accuracy, sensitivity, specificity, and BF-Score for lumen.

| Patient | Experiments | Accuracy | Sensitivity | Specificity | BF-Score |
|---|---|---|---|---|---|
| 0004 | AutoSeg/ | 0.97 ± 0.02 | 0.93 ± 0.05 | 0.99 ± 0.01 | 0.92 ± 0.09 |
| | Ground-truth | | | | |
| | AutoSeg/ | 0.96 ± 0.03 | 0.92 ± 0.05 | 0.99 ± 0.01 | 0.97 ± 0.05 |
| | Trained | | | | |
| | observer | | | | |
| | Ground-truth/ | 0.91 ± 0.05 | 0.82 ± 0.10 | 0.99 ± 0.01 | 0.97 ± 0.03 |
| | Trained | | | | |
| | observer | | | | |
| 0011 | AutoSeg/ | 0.97 ± 0.03 | 0.95 ± 0.05 | 1.00 ± 0.00 | 0.88 ± 0.09 |
| | Ground-truth | | | | |
| | AutoSeg/ | 0.96 ± 0.04 | 0.93 ± 0.07 | 0.99 ± 0.01 | 0.88 ± 0.10 |
| | Trained | | | | |
| | observer | | | | |
| | Ground-truth/ | 0.93 ± 0.07 | 0.87 ± 0.14 | 0.99 ± 0.01 | 0.98 ± 0.02 |
| | Trained | | | | |
| | observer | | | | |
| 0018 | AutoSeg/ | 0.97 ± 0.03 | 0.94 ± 0.06 | 0.99 ± 0.01 | 0.97 ± 0.03 |
| | Ground-truth | | | | |
| | AutoSeg/ | 0.96 ± 0.04 | 0.93 ± 0.07 | 0.99 ± 0.01 | 0.97 ± 0.03 |
| | Trained | | | | |
| | observer | | | | |
| | Ground-truth/ | 0.95 ± 0.05 | 0.89 ± 0.10 | 0.99 ± 0.01 | 0.98 ± 0.02 |
| | Trained | | | | |
| | observer | | | | |
| 0034 | AutoSeg/ | 0.95 ± 0.05 | 0.90 ± 0.09 | 0.99 ± 0.01 | 0.93 ± 0.07 |
| | Ground-truth | | | | |
| | AutoSeg/ | 0.96 ± 0.04 | 0.92 ± 0.08 | 0.99 ± 0.01 | 0.95 ± 0.05 |
| | Trained | | | | |
| | observer | | | | |
| | Ground-truth/ | 0.95 ± 0.03 | 0.91 ± 0.06 | 0.99 ± 0.01 | 0.98 ± 0.02 |
| | Trained | | | | |
| | observer | | | | |
| 0042 | AutoSeg/ | 0.96 ± 0.03 | 0.91 ± 0.06 | 0.99 ± 0.01 | 0.93 ± 0.07 |
| | Ground-truth | | | | |
| | AutoSeg/ | 0.97 ± 0.03 | 0.93 ± 0.06 | 0.99 ± 0.01 | 0.95 ± 0.05 |
| | Trained | | | | |
| | observer | | | | |
| | Ground-truth/ | 0.94 ± 0.05 | 0.88 ± 0.10 | 0.99 ± 0.01 | 0.98 ± 0.02 |
| | Trained | | | | |
| | observer | | | | |
| 0043 | AutoSeg/ | 0.94 ± 0.05 | 0.90 ± 0.09 | 0.99 ± 0.01 | 0.93 ± 0.07 |
| | Ground-truth | | | | |
| | AutoSeg/ | 0.95 ± 0.03 | 0.90 ± 0.07 | 0.99 ± 0.01 | 0.94 ± 0.06 |
| | Trained | | | | |
| | observer | | | | |
| | Ground-truth/ | 0.94 ± 0.05 | 0.88 ± 0.10 | 0.99 ± 0.01 | 0.98 ± 0.02 |
| | Trained | | | | |
| | observer | | | | |
| 0060 | AutoSeg/ | 0.95 ± 0.04 | 0.90 ± 0.09 | 0.99 ± 0.01 | 0.96 ± 0.04 |
| | Ground-truth | | | | |
| | AutoSeg/ | 0.95 ± 0.03 | 0.90 ± 0.07 | 0.99 ± 0.01 | 0.98 ± 0.02 |
| | Trained | | | | |
| | observer | | | | |
| | Ground-truth/ | 0.92 ± 0.07 | 0.84 ± 0.15 | 0.99 ± 0.01 | 0.98 ± 0.02 |
| | Trained | | | | |
| | observer | | | | |
| 0061 | AutoSeg/ | 0.93 ± 0.05 | 0.86 ± 0.09 | 0.99 ± 0.01 | 0.97 ± 0.03 |
| | Ground-truth | | | | |
| | AutoSeg/ | 0.93 ± 0.03 | 0.86 ± 0.07 | 0.99 ± 0.01 | 0.98 ± 0.01 |
| | Trained | | | | |
| | observer | | | | |
| | Ground-truth/ | 0.95 ± 0.04 | 0.89 ± 0.09 | 0.99 ± 0.01 | 0.97 ± 0.03 |
| | Trained | | | | |
| | observer | | | | |
| 0062 | AutoSeg/ | 0.95 ± 0.04 | 0.90 ± 0.09 | 0.99 ± 0.01 | 0.87 ± 0.12 |
| | Ground-truth | | | | |
| | AutoSeg/ | 0.95 ± 0.05 | 0.91 ± 0.09 | 0.99 ± 0.01 | 0.88 ± 0.09 |
| | Trained | | | | |
| | observer | | | | |
| | Ground-truth/ | 0.94 ± 0.06 | 0.88 ± 0.12 | 0.99 ± 0.01 | 0.97 ± 0.03 |
| | Trained | | | | |
| | observer | | | | |
| 0063 | AutoSeg/ | 0.95 ± 0.04 | 0.91 ± 0.08 | 0.99 ± 0.01 | 0.86 ± 0.09 |
| | Ground-truth | | | | |
| | AutoSeg/ | 0.95 ± 0.05 | 0.90 ± 0.09 | 0.99 ± 0.01 | 0.88 ± 0.10 |
| | Trained | | | | |
| | observer | | | | |
| | Ground-truth/ | 0.92 ± 0.06 | 0.84 ± 0.15 | 0.99 ± 0.01 | 0.98 ± 0.02 |
| | Trained | | | | |
| | observer | | | | |
| 0064 | AutoSeg/ | 0.97 ± 0.01 | 0.94 ± 0.03 | 0.99 ± 0.01 | 0.94 ± 0.06 |
| | Ground-truth | | | | |
| | AutoSeg/ | 0.98 ± 0.01 | 0.97 ± 0.01 | 1.00 ± 0.00 | 0.95 ± 0.05 |
| | Trained | | | | |
| | observer | | | | |
| | Ground-truth/ | 0.96 ± 0.03 | 0.92 ± 0.05 | 0.99 ± 0.01 | 0.99 ± 0.01 |
| | Trained | | | | |
| | observer | | | | |
| 0065 | AutoSeg/ | 0.97 ± 0.03 | 0.94 ± 0.04 | 0.99 ± 0.01 | 0.96 ± 0.04 |
| | Ground-truth | | | | |
| | AutoSeg/ | 0.98 ± 0.01 | 0.96 ± 0.03 | 1.00 ± 0.00 | 0.98 ± 0.02 |
| | Trained | | | | |
| | observer | | | | |
| | Ground-truth/ | 0.95 ± 0.04 | 0.90 ± 0.09 | 0.99 ± 0.01 | 0.98 ± 0.02 |
| | Trained | | | | |
| | observer | | | | |
| 0066 | AutoSeg/ | 0.97 ± 0.03 | 0.95 ± 0.05 | 0.99 ± 0.01 | 0.75 ± 0.10 |
| | Ground-truth | | | | |
| | AutoSeg/ | 0.98 ± 0.02 | 0.96 ± 0.04 | 0.99 ± 0.01 | 0.81 ± 0.12 |
| | Trained | | | | |
| | observer | | | | |
| | Ground-truth/ | 0.95 ± 0.04 | 0.90 ± 0.08 | 0.99 ± 0.01 | 0.95 ± 0.05 |
| | Trained | | | | |
| | observer | | | | |
| 0067 | AutoSeg/ | 0.95 ± 0.04 | 0.91 ± 0.08 | 0.99 ± 0.01 | 0.95 ± 0.05 |
| | Ground-truth | | | | |
| | AutoSeg/ | 0.96 ± 0.03 | 0.93 ± 0.07 | 0.99 ± 0.01 | 0.96 ± 0.04 |
| | Trained | | | | |
| | observer | | | | |
| | Ground-truth/ | 0.95 ± 0.05 | 0.89 ± 0.10 | 0.99 ± 0.01 | 0.98 ± 0.02 |
| | Trained | | | | |
| | observer | | | | |
| 0068 | AutoSeg/ | 0.94 ± 0.04 | 0.88 ± 0.08 | 0.99 ± 0.01 | 0.99 ± 0.01 |
| | Ground-truth | | | | |
| | AutoSeg/ | 0.94 ± 0.03 | 0.90 ± 0.07 | 0.99 ± 0.01 | 0.99 ± 0.01 |
| | Trained | | | | |
| | observer | | | | |
| | Ground-truth/ | 0.94 ± 0.06 | 0.87 ± 0.12 | 0.99 ± 0.01 | 0.99 ± 0.01 |
| | Trained | | | | |
| | observer | | | | |
| 0069 | AutoSeg/ | 0.96 ± 0.03 | 0.92 ± 0.06 | 0.99 ± 0.01 | 0.98 ± 0.02 |
| | Ground-truth | | | | |
| | AutoSeg/ | 0.97 ± 0.02 | 0.94 ± 0.05 | 0.99 ± 0.01 | 0.99 ± 0.01 |
| | Trained | | | | |
| | observer | | | | |
| | Ground-truth/ | 0.96 ± 0.03 | 0.92 ± 0.06 | 0.99 ± 0.01 | 0.99 ± 0.01 |
| | Trained | | | | |
| | observer | | | | |
| 0070 | AutoSeg/ | 0.97 ± 0.03 | 0.93 ± 0.07 | 0.99 ± 0.01 | 0.98 ± 0.02 |
| | Ground-truth | | | | |
| | AutoSeg/ | 0.97 ± 0.03 | 0.94 ± 0.06 | 0.99 ± 0.01 | 0.99 ± 0.01 |
| | Trained | | | | |
| | observer | | | | |
| | Ground-truth/ | 0.94 ± 0.06 | 0.88 ± 0.12 | 0.99 ± 0.01 | 0.99 ± 0.01 |
| | Trained | | | | |
| | observer | | | | |
| 0072 | AutoSeg/ | 0.97 ± 0.03 | 0.94 ± 0.05 | 1.00 ± 0.00 | 0.96 ± 0.04 |
| | Ground-truth | | | | |
| | AutoSeg/ | 0.98 ± 0.02 | 0.96 ± 0.04 | 1.00 ± 0.00 | 0.97 ± 0.03 |
| | Trained | | | | |
| | observer | | | | |
| | Ground-truth/ | 0.96 ± 0.03 | 0.91 ± 0.06 | 0.99 ± 0.01 | 0.98 ± 0.02 |
| | Trained | | | | |
| | observer | | | | |
| Pro- | AutoSeg/ | 0.97 ± 0.03 | 0.94 ± 0.06 | 0.99 ± 0.01 | 0.96 ± 0.04 |
| spective | Ground-truth | | | | |
| 4 | AutoSeg/ | 0.97 ± 0.03 | 0.95 ± 0.05 | 0.99 ± 0.01 | 0.98 ± 0.02 |
| | Trained | | | | |
| | observer | | | | |
| | Ground-truth/ | 0.94 ± 0.05 | 0.87 ± 0.10 | 0.99 ± 0.01 | 0.98 ± 0.02 |
| | Trained | | | | |
| | observer | | | | |

TABLE 5
Measured accuracy, sensitivity, specificity, and BF-Score for ROI
including the aortic wall, lumen, ILT, and calcification.

| Patient | Experiments | Accuracy | Sensitivity | Specificity | BF-Score |
|---|---|---|---|---|---|
| 0004 | AutoSeg/ | 0.96 ± 0.04 | 0.93 ± 0.07 | 0.99 ± 0.01 | 0.93 ± 0.09 |
| | Ground-truth | | | | |
| | AutoSeg/ | 0.98 ± 0.02 | 0.96 ± 0.04 | 1.00 ± 0.00 | 0.95 ± 0.05 |
| | Trained | | | | |
| | observer | | | | |
| | Ground-truth/ | 0.96 ± 0.03 | 0.93 ± 0.06 | 0.99 ± 0.01 | 0.98 ± 0.02 |
| | Trained | | | | |
| | observer | | | | |
| 0011 | AutoSeg/ | 0.93 ± 0.05 | 0.85 ± 0.09 | 0.99 ± 0.01 | 0.91 ± 0.09 |
| | Ground-truth | | | | |
| | AutoSeg/ | 0.94 ± 0.05 | 0.87 ± 0.09 | 0.99 ± 0.01 | 0.91 ± 0.09 |
| | Trained | | | | |
| | observer | | | | |
| | Ground-truth/ | 0.97 ± 0.03 | 0.95 ± 0.05 | 0.99 ± 0.01 | 0.98 ± 0.02 |
| | Trained | | | | |
| | observer | | | | |
| 0018 | AutoSeg/ | 0.95 ± 0.04 | 0.90 ± 0.08 | 0.99 ± 0.01 | 0.97 ± 0.03 |
| | Ground-truth | | | | |
| | AutoSeg/ | 0.95 ± 0.05 | 0.89 ± 0.08 | 0.99 ± 0.01 | 0.97 ± 0.03 |
| | Trained | | | | |
| | observer | | | | |
| | Ground-truth/ | 0.96 ± 0.03 | 0.93 ± 0.07 | 0.99 ± 0.01 | 0.99 ± 0.01 |
| | Trained | | | | |
| | observer | | | | |
| 0034 | AutoSeg/ | 0.95 ± 0.05 | 0.90 ± 0.08 | 0.99 ± 0.01 | 0.92 ± 0.08 |
| | Ground-truth | | | | |
| | AutoSeg/ | 0.97 ± 0.03 | 0.95 ± 0.05 | 1.00 ± 0.00 | 0.93 ± 0.07 |
| | Trained | | | | |
| | observer | | | | |
| | Ground-truth/ | 0.98 ± 0.02 | 0.97 ± 0.03 | 1.00 ± 0.00 | 0.98 ± 0.02 |
| | Trained | | | | |
| | observer | | | | |
| 0042 | AutoSeg/ | 0.92 ± 0.06 | 0.83 ± 0.09 | 0.99 ± 0.01 | 0.90 ± 0.09 |
| | Ground-truth | | | | |
| | AutoSeg/ | 0.94 ± 0.06 | 0.88 ± 0.10 | 0.99 ± 0.01 | 0.91 ± 0.09 |
| | Trained | | | | |
| | observer | | | | |
| | Ground-truth/ | 0.97 ± 0.02 | 0.95 ± 0.05 | 0.99 ± 0.01 | 0.98 ± 0.02 |
| | Trained | | | | |
| | observer | | | | |
| 0043 | AutoSeg/ | 0.95 ± 0.05 | 0.91 ± 0.09 | 0.99 ± 0.01 | 0.91 ± 0.09 |
| | Ground-truth | | | | |
| | AutoSeg/ | 0.98 ± 0.02 | 0.96 ± 0.04 | 1.00 ± 0.00 | 0.92 ± 0.10 |
| | Trained | | | | |
| | observer | | | | |
| | Ground-truth/ | 0.97 ± 0.03 | 0.94 ± 0.06 | 0.99 ± 0.00 | 0.95 ± 0.05 |
| | Trained | | | | |
| | observer | | | | |
| 0060 | AutoSeg/ | 0.95 ± 0.05 | 0.91 ± 0.09 | 0.99 ± 0.01 | 0.96 ± 0.04 |
| | Ground-truth | | | | |
| | AutoSeg/ | 0.98 ± 0.02 | 0.96 ± 0.04 | 1.00 ± 0.00 | 0.98 ± 0.02 |
| | Trained | | | | |
| | observer | | | | |
| | Ground-truth/ | 0.97 ± 0.03 | 0.95 ± 0.05 | 0.99 ± 0.01 | 0.98 ± 0.02 |
| | Trained | | | | |
| | observer | | | | |
| 0061 | AutoSeg/ | 0.94 ± 0.04 | 0.90 ± 0.08 | 0.99 ± 0.01 | 0.98 ± 0.02 |
| | Ground-truth | | | | |
| | AutoSeg/ | 0.96 ± 0.04 | 0.93 ± 0.04 | 0.99 ± 0.01 | 0.98 ± 0.02 |
| | Trained | | | | |
| | observer | | | | |
| | Ground-truth/ | 0.98 ± 0.02 | 0.96 ± 0.04 | 1.00 ± 0.00 | 0.99 ± 0.01 |
| | Trained | | | | |
| | observer | | | | |
| 0062 | AutoSeg/ | 0.90 ± 0.07 | 0.80 ± 0.09 | 0.99 ± 0.01 | 0.86 ± 0.11 |
| | Ground-truth | | | | |
| | AutoSeg/ | 0.92 ± 0.07 | 0.84 ± 0.09 | 0.99 ± 0.01 | 0.87 ± 0.09 |
| | Trained | | | | |
| | observer | | | | |
| | Ground-truth/ | 0.98 ± 0.02 | 0.96 ± 0.04 | 1.00 ± 0.00 | 0.98 ± 0.02 |
| | Trained | | | | |
| | observer | | | | |
| 0063 | AutoSeg/ | 0.92 ± 0.04 | 0.85 ± 0.09 | 0.99 ± 0.01 | 0.90 ± 0.09 |
| | Ground-truth | | | | |
| | AutoSeg/ | 0.94 ± 0.05 | 0.88 ± 0.10 | 0.99 ± 0.01 | 0.90 ± 0.09 |
| | Trained | | | | |
| | observer | | | | |
| | Ground-truth/ | 0.97 ± 0.03 | 0.95 ± 0.05 | 0.99 ± 0.01 | 0.98 ± 0.02 |
| | Trained | | | | |
| | observer | | | | |
| 0064 | AutoSeg/ | 0.91 ± 0.06 | 0.83 ± 0.10 | 0.99 ± 0.01 | 0.85 ± 0.09 |
| | Ground-truth | | | | |
| | AutoSeg/ | 0.94 ± 0.06 | 0.88 ± 0.10 | 0.99 ± 0.01 | 0.86 ± 0.10 |
| | Trained | | | | |
| | observer | | | | |
| | Ground-truth/ | 0.99 ± 0.01 | 0.97 ± 0.02 | 1.00 ± 0.00 | 0.98 ± 0.02 |
| | Trained | | | | |
| | observer | | | | |
| 0065 | AutoSeg/ | 0.97 ± 0.02 | 0.94 ± 0.04 | 0.99 ± 0.01 | 0.96 ± 0.04 |
| | Ground-truth | | | | |
| | AutoSeg/ | 0.99 ± 0.01 | 0.98 ± 0.02 | 1.00 ± 0.00 | 0.96 ± 0.04 |
| | Trained | | | | |
| | observer | | | | |
| | Ground-truth/ | 0.99 ± 0.01 | 0.97 ± 0.03 | 1.00 ± 0.00 | 0.97 ± 0.03 |
| | Trained | | | | |
| | observer | | | | |
| 0066 | AutoSeg/ | 0.95 ± 0.05 | 0.90 ± 0.09 | 0.99 ± 0.01 | 0.81 ± 0.10 |
| | Ground-truth | | | | |
| | AutoSeg/ | 0.96 ± 0.04 | 0.93 ± 0.07 | 0.99 ± 0.01 | 0.82 ± 0.10 |
| | Trained | | | | |
| | observer | | | | |
| | Ground-truth/ | 0.98 ± 0.02 | 0.96 ± 0.04 | 1.00 ± 0.00 | 0.96 ± 0.04 |
| | Trained | | | | |
| | observer | | | | |
| 0067 | AutoSeg/ | 0.92 ± 0.05 | 0.84 ± 0.09 | 0.99 ± 0.01 | 0.95 ± 0.05 |
| | Ground-truth | | | | |
| | AutoSeg/ | 0.96 ± 0.03 | 0.91 ± 0.06 | 0.99 ± 0.01 | 0.96 ± 0.04 |
| | Trained | | | | |
| | observer | | | | |
| | Ground-truth/ | 0.98 ± 0.02 | 0.96 ± 0.04 | 1.00 ± 0.00 | 0.99 ± 0.01 |
| | Trained | | | | |
| | observer | | | | |
| 0068 | AutoSeg/ | 0.96 ± 0.02 | 0.91 ± 0.05 | 0.99 ± 0.01 | 0.99 ± 0.01 |
| | Ground-truth | | | | |
| | AutoSeg/ | 0.98 ± 0.02 | 0.96 ± 0.04 | 1.00 ± 0.00 | 0.99 ± 0.01 |
| | Trained | | | | |
| | observer | | | | |
| | Ground-truth/ | 0.98 ± 0.02 | 0.97 ± 0.03 | 1.00 ± 0.00 | 0.99 ± 0.01 |
| | Trained | | | | |
| | observer | | | | |
| 0069 | AutoSeg/ | 0.95 ± 0.04 | 0.91 ± 0.08 | 0.99 ± 0.01 | 0.97 ± 0.03 |
| | Ground-truth | | | | |
| | AutoSeg/ | 0.96 ± 0.03 | 0.93 ± 0.07 | 0.99 ± 0.01 | 0.98 ± 0.02 |
| | Trained | | | | |
| | observer | | | | |
| | Ground-truth/ | 0.97 ± 0.03 | 0.92 ± 0.08 | 0.99 ± 0.01 | 0.99 ± 0.01 |
| | Trained | | | | |
| | observer | | | | |
| 0070 | AutoSeg/ | 0.95 ± 0.03 | 0.91 ± 0.06 | 0.99 ± 0.01 | 0.98 ± 0.02 |
| | Ground-truth | | | | |
| | AutoSeg/ | 0.96 ± 0.03 | 0.92 ± 0.07 | 0.99 ± 0.01 | 0.99 ± 0.01 |
| | Trained | | | | |
| | observer | | | | |
| | Ground-truth/ | 0.96 ± 0.04 | 0.92 ± 0.08 | 0.99 ± 0.01 | 0.99 ± 0.01 |
| | Trained | | | | |
| | observer | | | | |
| 0072 | AutoSeg/ | 0.88 ± 0.07 | 0.76 ± 0.11 | 0.99 ± 0.01 | 0.82 ± 0.09 |
| | Ground-truth | | | | |
| | AutoSeg/ | 0.91 ± 0.06 | 0.82 ± 0.12 | 0.99 ± 0.01 | 0.85 ± 0.13 |
| | Trained | | | | |
| | observer | | | | |
| | Ground-truth/ | 0.98 ± 0.01 | 0.95 ± 0.04 | 0.99 ± 0.01 | 0.97 ± 0.03 |
| | Trained | | | | |
| | observer | | | | |
| Pro- | AutoSeg/ | 0.95 ± 0.05 | 0.90 ± 0.09 | 0.99 ± 0.01 | 0.94 ± 0.06 |
| spective4 | Ground-truth | | | | |
| | AutoSeg/ | 0.97 ± 0.03 | 0.94 ± 0.06 | 0.99 ± 0.01 | 0.95 ± 0.05 |
| | Trained | | | | |
| | observer | | | | |
| | Ground-truth/ | 0.97 ± 0.02 | 0.91 ± 0.09 | 0.99 ± 0.01 | 0.91 ± 0.09 |
| | Trained | | | | |
| | observer | | | | |

TABLE 6
Measured accuracy, sensitivity, specificity, and BF-Score for ROI
including the aortic wall, celiac, lumen, ILT, and calcification.

| Patient | Experiments | Accuracy | Sensitivity | Specificity | BF-Score |
|---|---|---|---|---|---|
| 0004 | AutoSeg/ | 0.96 ± 0.03 | 0.93 ± 0.07 | 0.99 ± 0.01 | 0.91 ± 0.09 |
| | Ground- | | | | |
| | truth | | | | |
| | AutoSeg/ | 0.98 ± 0.02 | 0.96 ± 0.04 | 1.00 ± 0.00 | 0.94 ± 0.06 |
| | Trained | | | | |
| | observer | | | | |
| | Ground- | 0.96 ± 0.03 | 0.92 ± 0.06 | 0.99 ± 0.01 | 0.98 ± 0.02 |
| | truth/Trained | | | | |
| | observer | | | | |
| 0011 | AutoSeg/ | 0.93 ± 0.04 | 0.87 ± 0.08 | 0.99 ± 0.01 | 0.92 ± 0.09 |
| | Ground- | | | | |
| | truth | | | | |
| | AutoSeg/ | 0.95 ± 0.03 | 0.90 ± 0.07 | 0.99 ± 0.01 | 0.94 ± 0.06 |
| | Trained | | | | |
| | observer | | | | |
| | Ground- | 0.97 ± 0.03 | 0.95 ± 0.05 | 1.00 ± 0.00 | 0.98 ± 0.02 |
| | truth/Trained | | | | |
| | observer | | | | |
| 0018 | AutoSeg/ | 0.95 ± 0.03 | 0.90 ± 0.07 | 0.99 ± 0.01 | 0.96 ± 0.04 |
| | Ground- | | | | |
| | truth | | | | |
| | AutoSeg/ | 0.95 ± 0.03 | 0.90 ± 0.07 | 0.99 ± 0.01 | 0.95 ± 0.05 |
| | Trained | | | | |
| | observer | | | | |
| | Ground- | 0.96 ± 0.03 | 0.93 ± 0.07 | 0.99 ± 0.01 | 0.99 ± 0.01 |
| | truth/Trained | | | | |
| | observer | | | | |
| 0034 | AutoSeg/ | 0.95 ± 0.04 | 0.90 ± 0.09 | 0.99 ± 0.01 | 0.88 ± 0.09 |
| | Ground- | | | | |
| | truth | | | | |
| | AutoSeg/ | 0.98 ± 0.02 | 0.96 ± 0.04 | 1.00 ± 0.00 | 0.90 ± 0.09 |
| | Trained | | | | |
| | observer | | | | |
| | Ground- | 0.98 ± 0.02 | 0.97 ± 0.03 | 1.00 ± 0.00 | 0.98 ± 0.02 |
| | truth/Trained | | | | |
| | observer | | | | |
| 0042 | AutoSeg/ | 0.91 ± 0.08 | 0.81 ± 0.09 | 0.99 ± 0.01 | 0.87 ± 0.09 |
| | Ground- | | | | |
| | truth | | | | |
| | AutoSeg/ | 0.93 ± 0.06 | 0.86 ± 0.10 | 0.99 ± 0.01 | 0.88 ± 0.10 |
| | Trained | | | | |
| | observer | | | | |
| | Ground- | 0.97 ± 0.03 | 0.95 ± 0.05 | 0.99 ± 0.01 | 0.98 ± 0.02 |
| | truth/Trained | | | | |
| | observer | | | | |
| 0043 | AutoSeg/ | 0.95 ± 0.05 | 0.91 ± 0.09 | 0.99 ± 0.01 | 0.87 ± 0.09 |
| | Ground- | | | | |
| | truth | | | | |
| | AutoSeg/ | 0.98 ± 0.02 | 0.95 ± 0.05 | 1.00 ± 0.00 | 0.90 ± 0.10 |
| | Trained | | | | |
| | observer | | | | |
| | Ground- | 0.97 ± 0.03 | 0.94 ± 0.06 | 0.99 ± 0.01 | 0.95 ± 0.05 |
| | truth/Trained | | | | |
| | observer | | | | |
| 0060 | AutoSeg/ | 0.95 ± 0.05 | 0.90 ± 0.09 | 0.99 ± 0.01 | 0.93 ± 0.07 |
| | Ground- | | | | |
| | truth | | | | |
| | AutoSeg/ | 0.98 ± 0.02 | 0.96 ± 0.04 | 1.00 ± 0.00 | 0.94 ± 0.06 |
| | Trained | | | | |
| | observer | | | | |
| | Ground- | 0.97 ± 0.03 | 0.95 ± 0.05 | 0.99 ± 0.01 | 0.98 ± 0.02 |
| | truth/Trained | | | | |
| | observer | | | | |
| 0061 | AutoSeg/ | 0.93 ± 0.04 | 0.89 ± 0.08 | 0.99 ± 0.01 | 0.96 ± 0.04 |
| | Ground- | | | | |
| | truth | | | | |
| | AutoSeg/ | 0.96 ± 0.03 | 0.93 ± 0.07 | 0.99 ± 0.01 | 0.97 ± 0.03 |
| | Trained | | | | |
| | observer | | | | |
| | Ground- | 0.98 ± 0.02 | 0.96 ± 0.04 | 1.00 ± 0.00 | 0.99 ± 0.01 |
| | truth/Trained | | | | |
| | observer | | | | |
| 0062 | AutoSeg/ | 0.90 ± 0.06 | 0.80 ± 0.10 | 0.99 ± 0.01 | 0.83 ± 0.11 |
| | Ground- | | | | |
| | truth | | | | |
| | AutoSeg/ | 0.91 ± 0.07 | 0.83 ± 0.09 | 0.99 ± 0.01 | 0.83 ± 0.09 |
| | Trained | | | | |
| | observer | | | | |
| | Ground- | 0.98 ± 0.02 | 0.96 ± 0.04 | 1.00 ± 0.00 | 0.98 ± 0.02 |
| | truth/Trained | | | | |
| | observer | | | | |
| 0063 | AutoSeg/ | 0.92 ± 0.04 | 0.85 ± 0.08 | 0.99 ± 0.01 | 0.90 ± 0.09 |
| | Ground- | | | | |
| | truth | | | | |
| | AutoSeg/ | 0.94 ± 0.05 | 0.88 ± 0.09 | 0.99 ± 0.01 | 0.90 ± 0.09 |
| | Trained | | | | |
| | observer | | | | |
| | Ground- | 0.97 ± 0.03 | 0.95 ± 0.05 | 0.99 ± 0.01 | 0.98 ± 0.02 |
| | truth/Trained | | | | |
| | observer | | | | |
| 0064 | AutoSeg/ | 0.92 ± 0.04 | 0.84 ± 0.06 | 0.99 ± 0.01 | 0.86 ± 0.09 |
| | Ground- | | | | |
| | truth | | | | |
| | AutoSeg/ | 0.95 ± 0.04 | 0.90 ± 0.09 | 0.99 ± 0.01 | 0.87 ± 0.09 |
| | Trained | | | | |
| | observer | | | | |
| | Ground- | 0.99 ± 0.01 | 0.97 ± 0.02 | 1.00 ± 0.00 | 0.98 ± 0.02 |
| | truth/Trained | | | | |
| | observer | | | | |
| 0065 | AutoSeg/ | 0.97 ± 0.02 | 0.94 ± 0.06 | 0.99 ± 0.01 | 0.93 ± 0.07 |
| | Ground- | | | | |
| | truth | | | | |
| | AutoSeg/ | 0.99 ± 0.01 | 0.98 ± 0.02 | 1.00 ± 0.00 | 0.91 ± 0.09 |
| | Trained | | | | |
| | observer | | | | |
| | Ground- | 0.99 ± 0.01 | 0.97 ± 0.03 | 1.00 ± 0.00 | 0.97 ± 0.03 |
| | truth/Trained | | | | |
| | observer | | | | |
| 0066 | AutoSeg/ | 0.94 ± 0.04 | 0.90 ± 0.09 | 0.99 ± 0.01 | 0.81 ± 0.10 |
| | Ground- | | | | |
| | truth | | | | |
| | AutoSeg/ | 0.95 ± 0.05 | 0.91 ± 0.09 | 0.99 ± 0.01 | 0.80 ± 0.10 |
| | Trained | | | | |
| | observer | | | | |
| | Ground- | 0.98 ± 0.02 | 0.96 ± 0.04 | 0.99 ± 0.01 | 0.96 ± 0.04 |
| | truth/Trained | | | | |
| | observer | | | | |
| 0067 | AutoSeg | 0.93 ± 0.04 | 0.86 ± 0.08 | 0.99 ± 0.01 | 0.93 ± 0.09 |
| | Ground- | | | | |
| | truth | | | | |
| | AutoSeg/ | 0.96 ± 0.02 | 0.92 ± 0.05 | 0.99 ± 0.01 | 0.94 ± 0.06 |
| | Trained | | | | |
| | observer | | | | |
| | Ground- | 0.98 ± 0.02 | 0.96 ± 0.04 | 1.00 ± 0.00 | 0.99 ± 0.01 |
| | truth/Trained | | | | |
| | observer | | | | |
| 0068 | AutoSeg | 0.95 ± 0.03 | 0.91 ± 0.07 | 0.99 ± 0.01 | 0.98 ± 0.02 |
| | Ground- | | | | |
| | truth | | | | |
| | AutoSeg/ | 0.98 ± 0.02 | 0.95 ± 0.05 | 1.00 ± 0.00 | 0.98 ± 0.02 |
| | Trained | | | | |
| | observer | | | | |
| | Ground- | 0.98 ± 0.02 | 0.97 ± 0.03 | 1.00 ± 0.00 | 0.99 ± 0.01 |
| | truth/Trained | | | | |
| | observer | | | | |
| 0069 | AutoSeg | 0.95 ± 0.03 | 0.91 ± 0.06 | 0.99 ± 0.01 | 0.98 ± 0.02 |
| | Ground- | | | | |
| | truth | | | | |
| | AutoSeg/ | 0.96 ± 0.03 | 0.93 ± 0.06 | 0.99 ± 0.01 | 0.99 ± 0.01 |
| | Trained | | | | |
| | observer | | | | |
| | Ground- | 0.97 ± 0.03 | 0.92 ± 0.08 | 0.99 ± 0.01 | 0.99 ± 0.01 |
| | truth/Trained | | | | |
| | observer | | | | |
| 0070 | AutoSeg/ | 0.95 ± 0.03 | 0.90 ± 0.07 | 0.99 ± 0.01 | 0.97 ± 0.03 |
| | Ground- | | | | |
| | truth | | | | |
| | AutoSeg/ | 0.95 ± 0.04 | 0.90 ± 0.08 | 0.99 ± 0.01 | 0.97 ± 0.03 |
| | Trained | | | | |
| | observer | | | | |
| | Ground- | 0.96 ± 0.04 | 0.92 ± 0.08 | 0.99 ± 0.01 | 0.99 ± 0.01 |
| | truth/Trained | | | | |
| | observer | | | | |
| 0072 | AutoSeg/ | 0.89 ± 0.07 | 0.78 ± 0.09 | 0.99 ± 0.01 | 0.84 ± 0.09 |
| | Ground- | | | | |
| | truth | | | | |
| | AutoSeg/ | 0.92 ± 0.06 | 0.84 ± 0.09 | 0.99 ± 0.01 | 0.87 ± 0.10 |
| | Trained | | | | |
| | observer | | | | |
| | Ground- | 0.96 ± 0.02 | 0.95 ± 0.04 | 0.98 ± 0.01 | 0.97 ± 0.03 |
| | truth/Trained | | | | |
| | observer | | | | |
| Pro- | AutoSeg/ | 0.94 ± 0.04 | 0.89 ± 0.09 | 0.99 ± 0.01 | 0.92 ± 0.08 |
| spective4 | Ground-truth | | | | |
| | AutoSeg/ | 0.96 ± 0.03 | 0.93 ± 0.06 | 0.99 ± 0.01 | 0.94 ± 0.06 |
| | Trained | | | | |
| | observer | | | | |
| | Ground- | 0.97 ± 0.02 | 0.91 ± 0.09 | 0.99 ± 0.01 | 0.91 ± 0.09 |
| | truth/Trained | | | | |
| | observer | | | | |

Modifications and improvements to the above-described implementations of the present technology may become apparent to those skilled in the art. The foregoing description is intended to be exemplary rather than limiting.

1. A method for segmenting aortic tissues in an image of a body of a given subject, the method being executed by a processor, the processor having access to at least one deep learning model having been trained to segment tissues in images, the method comprising:
receiving the image of the body of the given subject, the image comprising an aorta, an intraluminal thrombus and additional body parts;
extracting a region of interest from the received image, the region of interest comprising the aorta and the intraluminal thrombus;
determining, within the region of interest, a presence of a calcification on at least one of an aortic wall and the intraluminal thrombus; and
outputting an indication of the presence of the calcification.

2. The method of claim 1, wherein the at least one deep learning model comprises a first deep learning and a second deep learning model, said extracting the region of interest being performed by the first deep learning model and said determining the presence of the calcification is performed by the second deep learning model.

3. The method of claim 2, wherein said extracting the region of interest comprises:
extracting, using the first deep learning model, first image features from the image, the first image features being indicative of the aorta and the intraluminal thrombus; and
segmenting, using the first deep learning model, the region of interest from the image using the first image features.

4.-6. (canceled)

7. The method of claim 3, further comprising detecting an aortic lumen of the aorta within the region of interest, the aortic lumen, the aortic wall and the intraluminal thrombus forming together the region of interest.

8. (canceled)

9. (canceled)

10. The method of claim 7, wherein said detecting the aortic lumen is performed by the second deep learning model, said detecting the aortic lumen and said determining the presence of the calcification being concurrently performed by the second deep learning model.

11. The method of claim 10, wherein the second deep learning model is configured for extracting second image features from the region of interest, the second image features being indicative of the aortic lumen and the calcification, said determining the presence of the calcification being performed using the second image features.

12. (canceled)

13. The method of claim 7, wherein the at least one deep learning model further comprises a third deep learning model, said detecting the aortic lumen being performed by the third deep learning model, the method further comprising removing the aortic lumen from the region of interest, thereby obtaining the aortic wall and the intraluminal thrombus.

14. The method of claim 13, wherein said detecting the aortic lumen comprises:
extracting, using the third deep learning model, second image features from the region of interest, the second image features being indicative of the aortic lumen; and
segmenting, using the third deep learning model, the aortic lumen from the region of interest using the second image features; and
wherein the second deep learning model is configured for:
extracting third image features from the aortic wall and the intraluminal thrombus, the third image features being indicative of the calcification, said determining the presence of the calcification being performed by the second deep learning model using the third image features.

15. The method of claim 13, wherein the third deep learning model comprises a fully convolutional network (FCN)-based model.

16. The method of claim 15, wherein the third deep learning model comprises one of dilated convolutional layers and a binary classifier.

17.-20. (canceled)

21. A system for segmenting aortic tissues in images of a body of a given subject, the system comprising:
a processor;
a non-transitory storage medium operatively connected to the processor, the non-transitory storage medium comprising computer-readable instructions;
the processor having access to at least one deep learning model having been trained to segment tissues in images, the processor, upon executing the computer-readable instructions, being configured for:
receiving the image of the body of the given subject, the image comprising an aorta, an intraluminal thrombus and additional body parts;
extracting a region of interest from the received image, the region of interest comprising the aorta and the intraluminal thrombus;
determining, within the region of interest, a presence of a calcification on at least one of an aortic wall and the intraluminal thrombus; and
outputting an indication of the presence of the calcification.

22. The system of claim 21, wherein the at least one deep learning model comprises a first deep learning and a second deep learning model, said extracting the region of interest being performed by the first deep learning model and said determining the presence of the calcification is performed by the second deep learning model.

23. The system of claim 22, wherein said extracting the region of interest comprises:
extracting, using the first deep learning model, first image features from the image, the first image features being indicative of the aorta and the intraluminal thrombus; and
segmenting, using the first deep learning model, the region of interest from the image using the first image features.

24.-26. (canceled)

27. The system of claim 23, further comprising detecting an aortic lumen of the aorta within the region of interest, the aortic lumen, the aortic wall and the intraluminal thrombus forming together the region of interest.

28. (canceled)

29. (canceled)

30. The system of claim 27, wherein said detecting the aortic lumen is performed by the second deep learning model, said detecting the aortic lumen and said determining the presence of the calcification being concurrently performed by the second deep learning model.

31. The system of claim 30, wherein the second deep learning model is configured for extracting second image features from the region of interest, the second image features being indicative of the aortic lumen and the calcification, said determining the presence of the calcification being performed using the second image features.

32. (canceled)

33. The system of claim 27, wherein the at least one deep learning model further comprises a third deep learning model, said detecting the aortic lumen being performed by the third deep learning model, the method further comprising removing the aortic lumen from the region of interest, thereby obtaining the aortic wall and the intraluminal thrombus.

34. The system of claim 33, wherein said detecting the aortic lumen comprises:
extracting, using the third deep learning model, second image features from the region of interest, the second image features being indicative of the aortic lumen; and
segmenting, using the third deep learning model, the aortic lumen from the region of interest using the second image features; and
wherein the second deep learning model is configured for:
extracting third image features from the aortic wall and the intraluminal thrombus, the third image features being indicative of the calcification, said determining the presence of the calcification being performed by the second deep learning model using the third image features.

35. The system of claim 33, wherein the third deep learning model comprises a fully convolutional network (FCN)-based model.

36. The system of claim 35, wherein the third deep learning model comprises one of dilated convolutional layers and a binary classifier.

37.-40. (canceled)
